SEQUENCE LISTING (8 sequences)

SEQ ID NO: 1 - DNA, 20 bp, Artificial Sequence
  Synthetic Sequence
ctgaggaccggcaacatggt

SEQ ID NO: 2 - DNA, 20 bp, Artificial Sequence
  Synthetic Sequence
ccatcttccttgccaagtga

SEQ ID NO: 3 - DNA, 20 bp, Artificial Sequence
  Synthetic Sequence
aacacgtctcttgccaggtc

SEQ ID NO: 4 - DNA, 20 bp, Artificial Sequence
  Synthetic Sequence
cacaagtctggtggtggtgt

SEQ ID NO: 5 - DNA, 20 bp, Artificial Sequence
  Synthetic Sequence
ggaagtgcagcagggttcag

SEQ ID NO: 6 - DNA, 20 bp, Artificial Sequence
  Synthetic Sequence
ggttctgcttccctttcagt

SEQ ID NO: 7 - DNA, 20 bp, Artificial Sequence
  Synthetic Sequence
ccgctaagctggatgaaaga

SEQ ID NO: 8 - DNA, 20 bp, Artificial Sequence
  Synthetic Sequence
gactgggcatctatgctggc

(19) United States
(12) Patent Application Publication
(10) Pub. No.: US 2005/0158721 A1
(43) Pub. Date: Jul. 21, 2005

(54) Tissue rejection

(76) Inventor: Paul G Shiels, Glasgow (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/501,682

(22) PCT Filed: Jan. 16, 2003
(86) PCT No.: PCT/GB03/00187

(30) Foreign Application Priority Data
Jan. 16, 2002 (GB) 0200929.8

(51) Int. Cl. C12Q 1/68; G01N 33/53; G01N 33/567; A61K 48/00

(52) U.S. Cl. 435/6; 435/7.2; 514/44

(57) ABSTRACT

Methods and materials for treating tissues to reduce the risk of transplant rejection are described, along with methods for determining the risk of rejection of transplants. The level of expression of various senescence-associated genes, such as G22P1, XRCC5, hPOT1 and SIRT2 and homologues or analogues thereof, is considered indicative of tissue rejection, and their levels may be modulated to reduce the risk of rejection. The tissue are preferably renal tissues.

The present invention relates to a preparation for use in the evaluation of the risk of tissue rejection of donor tissues. The invention further relates to preparations for use in suppressing tissue rejection, and still further to methods of assessing and suppressing tissue rejection of donor tissues.

Transplantation is now the preferred treatment for cases of chronic renal failure. There is however a large disparity between the number of potential transplant recipients and the number of available donor kidneys. This disparity is also evident in all other transplant cases. Failure of transplanted or grafted tissues (the term ‘tissues’ as used herein will be understood to refer equally to cells and organs) is therefore of extreme clinical, economic, and emotional significance on recipients of donor tissue. In terms of kidney transplants, chronic allograft nephropathy (CAN) represents the most common cause of graft loss, once patient death has been accounted for. Unfortunately, the pathogenesis of CAN, at present, remains poorly understood and little progress has been made in reducing its occurrence. A better understanding of the pathogenesis of CAN may be expected to yield new light on how to reduce the occurrence of CAN (Halloran et al, 1999).

Previous hypotheses to explain the pathogenesis of CAN have concentrated on possible immune-based explanations. However, these now appear unlikely, since the majority of risk factors for CAN are non-immune, and the widespread introduction of new immunosuppressants in the 1980s did not lead to a significant drop in the occurrence of CAN (Paul, 1999).

It is among the objects of embodiments of the present invention to provide preparations for reducing the occurrence of CAN in donor tissues. This is achieved, in part, by the surprising findings of the present inventors that a number of senescence-associated genes (SAGs) have been found to show altered expression in tissues susceptible to CAN.

It has been suggested that one possible cause of CAN may be accelerated ageing, where the accumulated burden of injury and age exhausts the ability of key cells to repair and remodel, and therefore to retain tissue integrity (Halloran et al, 1999). The present invention is based upon a model for CAN in which damage to the tissue leads to altered expression of genes involved in senescence, predisposing to the development of CAN. The particular genes which are implicated in cellular senescence are the telomere-binding proteins, as discussed below. It has been surprisingly found by the present inventors that specific telomere-binding proteins show increased expression in kidneys that have been subject to CAN when compared to normal tissues. Surprisingly also, the present inventors have also found decreased expression of a given SAG in tissues derived from a cadaveric donor, when compared to those derived from a living donor, or indeed those from living donors that have subsequently undergone CAN.

According to a first aspect of the present invention, there is provided a method of screening mammalian donor tissues for predisposition to rejection, the method comprising the steps of determining the level of expression of an endogenous telomere-binding protein in the donor tissue, and comparing the determined level to a reference level of expression, altered levels of expression in the donor tissue being indicative of a predisposition to rejection.

Of course, it will be appreciated that the present method alone does not enable a medical practitioner to definitively assess whether or not a given donor tissue is suitable for transplant. Many other factors must be taken into consideration, for example, the age and general health of both the donor and the recipient, the urgency of the transplant, immunological factors, and so forth. The skilled person will be aware of the many relevant factors which will be considered. Nonetheless, the method of the present invention is intended to be a useful addition to the skilled person's other considerations and techniques.

Preferably the method is carried out on a donor tissue previously removed from a donor body. Alternatively, the method may be carried out on a donor tissue post-transplant; this may be used in order to provide data for the determination and suitability of therapeutic intervention or change of therapeutic regime.

The mammalian tissue may be human or animal tissue, such as porcine tissue.

In a particularly preferred embodiment the tissue being tested is renal tissue.

The method may comprise determining the expression level of one or more telomere binding proteins; preferably at least two, and more preferably at least three. This allows assessment to be made on the basis of a number of diagnostic proteins, to reduce the risk of false positives.

Preferably the telomere binding protein is selected from the group comprising G22P1, XRCC5, hPOT1, and SIRT2, and their homologues or analogues. These genes and their proteins are described and characterised in the publications listed in the OMIM database at:
G22P1—http://www3.ncbi.nlm.nih.gov/htbin-post/Omim/dispmim?152690
XRCC5—http://www3.ncbi.nlm.nih.gov/htbin-post/Omim/dispmim?194364
hPOT1—http://www3.ncbi.nlm.nih.gov/htbin-post/Omim/dispmim?606478
SIRT2—http://www3.ncbi.nlm.nih.gov/htbin-post/Omim/dispmim?604479

Where the telomere binding protein is one or more of G22P1, XRCC5, and hPOT1, and homologues thereof, an increase in expression of these proteins is taken as indicative of a predisposition to rejection. Where the telomere binding protein is SIRT2 or a homologue thereof, a decrease in expression of this protein is indicative of a predisposition to rejection in tissue taken from a cadaveric source.

The step of determining the level of expression of the telomere binding protein may be conducted by means of detection of the protein with antibodies thereto; the antibodies may themselves be labelled in some way (for example, radiolabelling, fluorescent labelling, or the like), or the antibodies may be subsequently detected using additional reagents, for example in the form of an ELISA assay well known to those of skill in the art. Alternatively, the levels of expression of mRNA encoding the protein may be determined, using, for example, Northern blotting, polymerase chain reaction (PCR) detection, in situ hybridisation, or the like.

The reference level of expression may be a predetermined reference level, or may be determined substantially at the same time as the donor tissue level of expression; for example, from tissues of the recipient or of a control subject. Preferably the reference level is obtained from a healthy tissue sample.

According to a second aspect of the present invention, there is provided a kit for screening mammalian donor tissues for predisposition to rejection, the kit comprising reagents for determining the level of expression of an endogenous telomere binding protein in the donor tissue.

Preferably the kit comprises PCR primers for detection of mRNA encoding the telomere binding protein. Preferably, the protein is selected from the group comprising G22P1, XRCC5, hPOT1, and SIRT2, or homologues and analogues thereof. In alternative embodiments of the invention, the protein may be derived from another telomere binding protein or senescence associated gene, as exemplified by and in no way limited by, Rif1, Rif2, Rap1, SIRTs1, 3, 4, 5, Est1, Est2, TLG1, cdc13, A26, ATM, HDAC1, hSEP1, hTEP1, HuCds1, MYC, NEK2, p21, PIN2, TNKS, TERC, hTERT, TOP2A, TOP2B, TP53, TRF1, TRF2, WRN. Full details of such genes are listed and linked to the following location http://www.genlink.wustl.edu/teldb/pteldb/pteldb3.html Alternatively, the kit may comprise antibodies to the telomere binding protein. The kit may further comprise detection reagents for detection of bound antibodies; alternatively or in addition the antibodies themselves may be labelled to allow direct or indirect detection thereof. For example, radioactive or fluorescent labels may be used. A further alternative for the kit is that it may provide probes (such as DNA, RNA, or chimerical combinations of both or either) together with a further agent, for example a proteinaceous agent such as an antibody or other agent.

According to a further aspect of the present invention, there is provided a method of treatment of a mammalian donor tissue to reduce the risk of rejection, the method comprising the step of treating the tissue with an agent to enhance the activity, half-life or expression level of an endogenous telomere binding protein. Alternatively, or in addition, the tissue may be treated with an agent to enhance the effective functionality of the endogenous telomere binding protein.

Alternatively, or in addition, the tissue may be treated with an agent to prevent tissue senescence or cell death. Such treatment is intended to prevent stress to a transplanted tissue, and the treatment may take place pre- or post-transplant. The agent may comprise a calcineurin inhibitor. Alternatively, analogues of such agents, for example small molecule inhibitors of the mitochondrial membrane permeability transition pore (MPTP), may be employed. These agents (calcineurin inhibitors, or their analogues) prevent apoptosis by directly or indirectly preventing calcium influx into the mitochondrion and apoptotic signalling as a consequence of the collapse of the mitochondrial membrane potential. This is discussed more fully in appendix 1 with reference to transplantation.

It is believed that the increased expression levels of certain telomere binding proteins reflects increased levels of cell damage in rejection-susceptible tissues. Without wishing to be bound by theory, it is believed that as cellular DNA is damaged, the telomere nucleoprotein complexes are disrupted and telomere erosion can occur. Increased expression of these proteins results as the cell attempts to maintain sufficient protein at the telomeres and facilitate damage responses; this increased expression is detectable as discussed above. However, if insufficient protein is present, telomere damage and cell death or degradation may occur. Thus, further increasing the expression or otherwise assisting the functionality of these proteins may be expected to act to repair the damage and so reduce the risk of tissue rejection.

The donor tissue may be human or animal.

Conveniently the tissue is renal tissue.

Preferably the protein is selected from the group comprising G22P1, XRCC5, hPOT1, and SIRT2, and homologues or analogues thereof. The protein may instead comprise an active site homologous to that of G22P1, XRCC5, hPOT1, or SIRT2. Alternatively the protein may derive from another SAG such as those in the following group, though it is in no way envisaged that this group is limiting: Rif1, Rif2, Rap1, SIRTs1, 3, 4, 5, Est1, Est2, TLG1, cdc13, A26, ATM, HDAC1, hSEP1, hTEP1, HuCds1, MYC, NEK2, p21, PIN2, TNKS, TERC, hTERT, TOP2A, TOP2B, TP53, TRF1, TRF2, WRN.

One or more protein activity, expression levels or half-life may be enhanced; preferably two or more; more preferably three or more proteins are enhanced.

The alteration of gene expression levels may be achieved via a number of strategies including: gene therapy; promoter/enhancer knock in; promoter/enhancer induction; gene replacement; interference RNA and the like. These methods are not considered an exhaustive list of the full range of such techniques which typically are known to those of skill in the art. In certain embodiments of the invention, the reduction of certain expression levels may be desired instead of or in addition to the enhancement of certain expression levels.

According to a further aspect of the present invention, there is provided the use of a mammalian telomere binding protein in the preparation of a medicament for suppression of rejection of a donor mammalian tissue.

The tissue is preferably renal tissue.

The protein is preferably selected from the group comprising G22P1, XRCC5, hPOT1, and SIRT2, and homologues and analogues thereof. Alternatively the protein may be selected from another SAG such as those in the following group, though it is in no way envisaged that this group is limiting: Rif1, Rif2, Rap1, SIRTs1, 3, 4, 5, Est1, Est2, TLG1, cdc13, A26, ATM, HDAC1, hSEP1, hTEP1, HuCds1, MYC, NEK2, p21, PIN2, TNKS, TERC, hTERT, TOP2A, TOP2B, TP53, TRF1, TRF2, WRN.

According to a still further aspect of the present invention, there is provided a non-human mammalian donor tissue in which the expression of at least one endogenous telomere binding protein has been enhanced.

Such tissues may be useful in xenotransplantation, to lessen the risk of rejection of the donor tissue. Enhancement may be as a result of gene therapy, or transgenesis or molecular manipulation of cells subsequently used for nuclear transplantation. Promoter or enhancer knock in or induction element knock in are also applicable for these purposes.

According to yet another aspect of the invention there is provided a method of assessing tissue damage based upon the accumulation of cytological markers of tissue stress. The markers may comprise the level of senescence associated beta galactosidase (SA beta gal, or SA β gal) at pH6, or the accumulation of lipofuscin, advanced glycation end products or iso-prostanes.

These and other aspects of the present invention will now be described with reference to the accompanying drawings, in which:

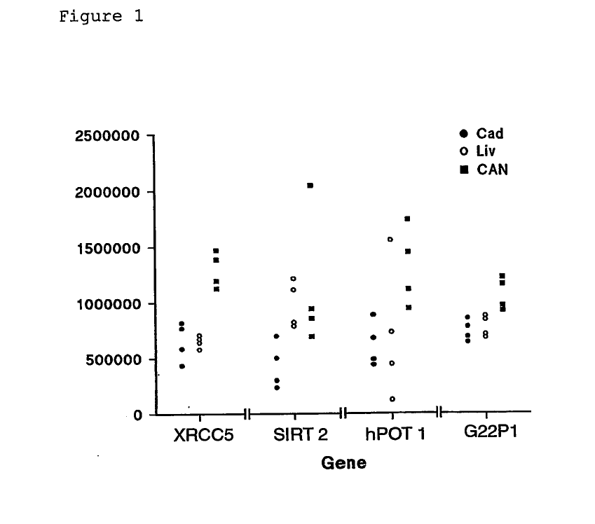

FIG. 1 shows the relative levels of expression of G22P1, XRCC5, hPOT1, and SIRT2 mRNA from different types of renal tissue;

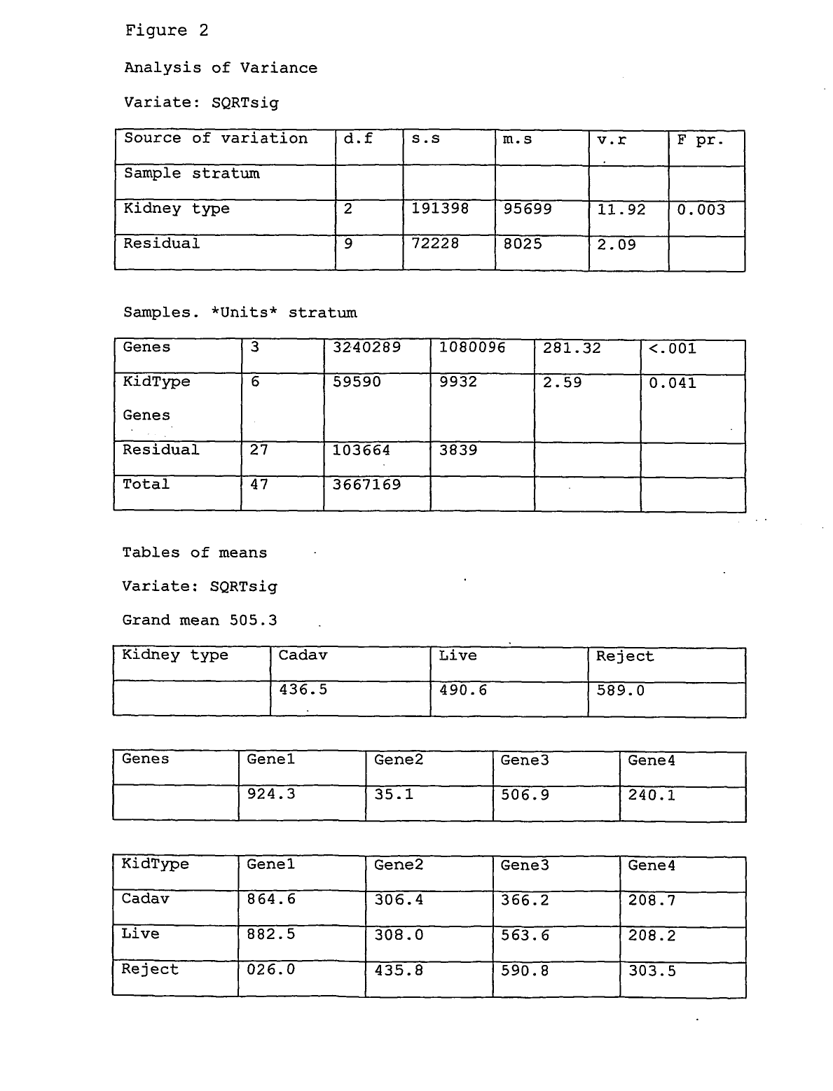

FIG. 2 shows the results of a statistical analysis of the results of FIG. 1;

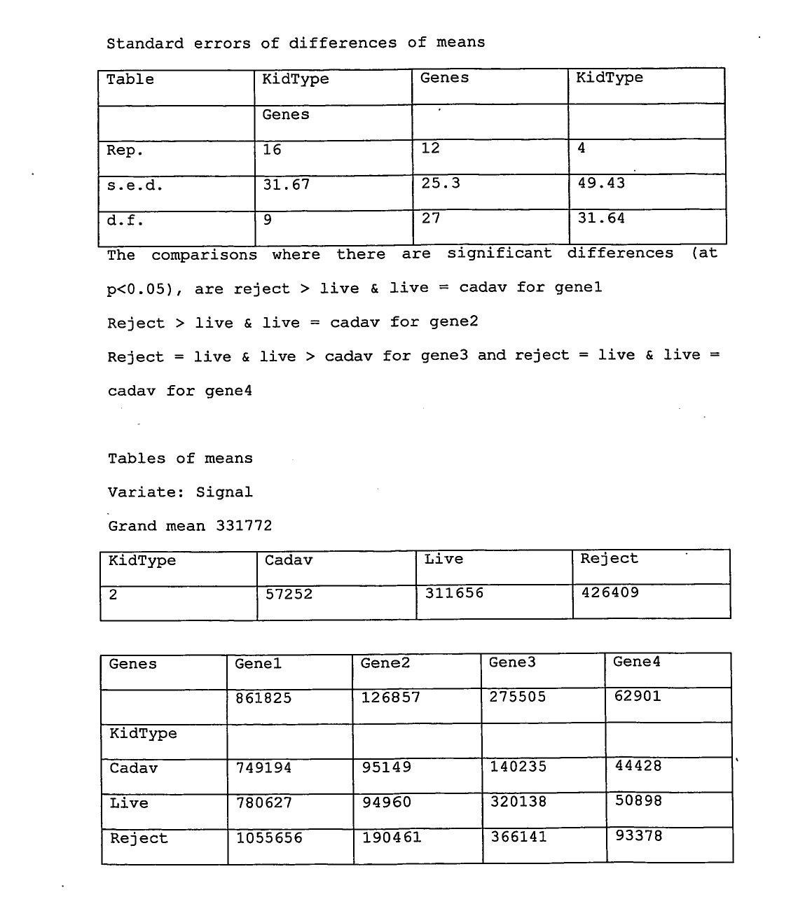

FIG. 3 shows the level of SA β gal staining at pH 6 in primary tubular epithelial cultures;

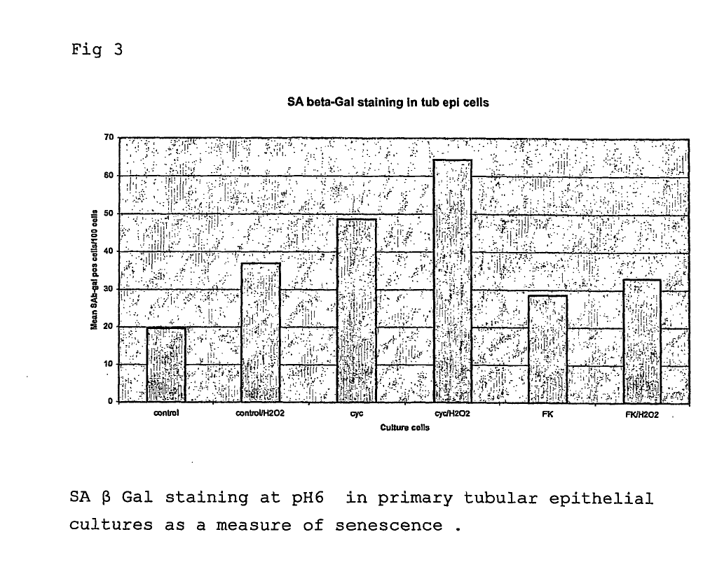

FIG. 4 shows a summary of cellular stress responses to oxidative damage;

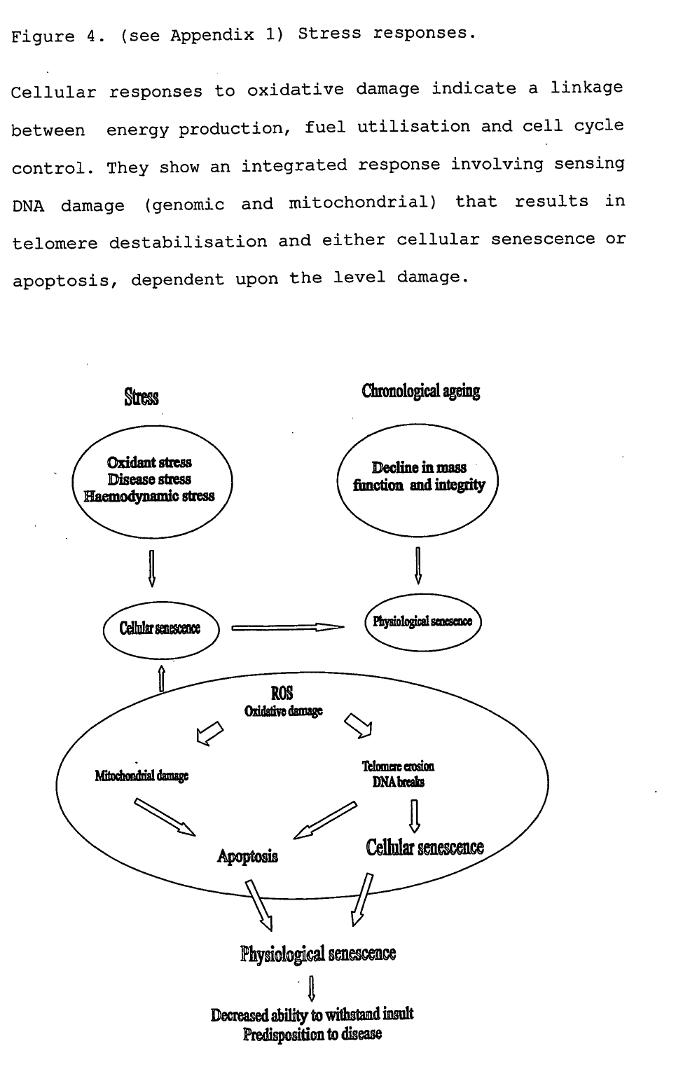

FIG. 5 shows a summary of cellular paths to senescence;

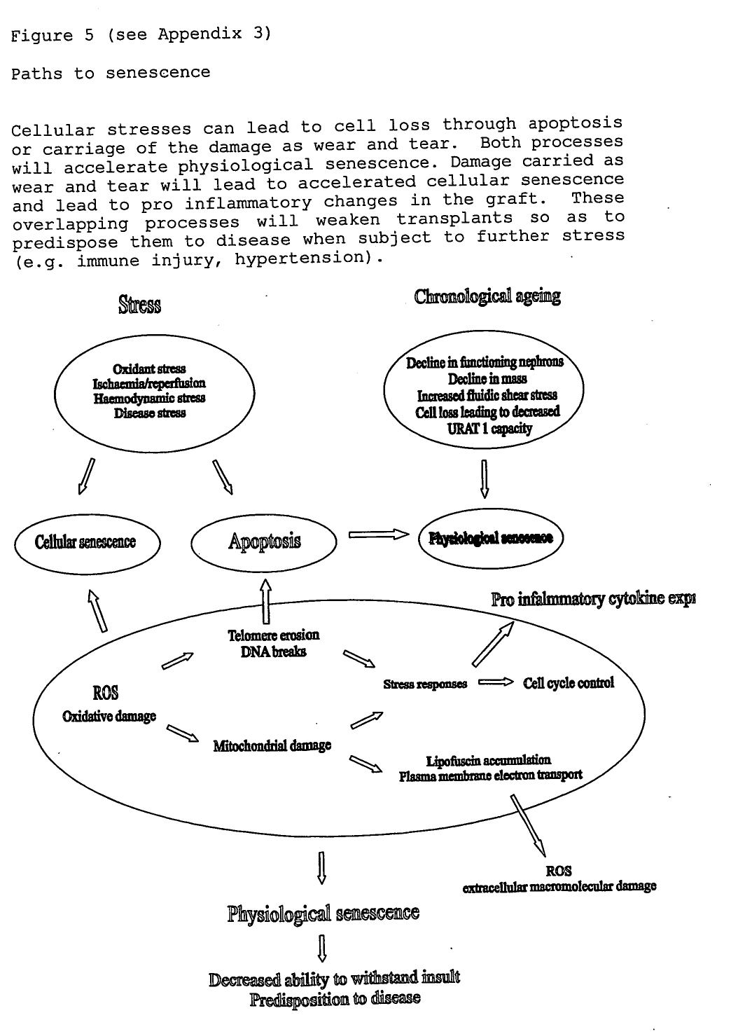

FIG. 6 shows tubular atrophy in F344 to LEW renal allografts at day 7, 14, 30, 60 and 100 post transplant according to Banff criteria (0: no tubular atrophy; 1: tubular atrophy in <25% of cortical tubules; 2: tubular atrophy in 26-50% of cortical tubules; 3: tubular atrophy in >51% of cortical tubules). LEW to F344 allografts are shown in day 60 post transplant;

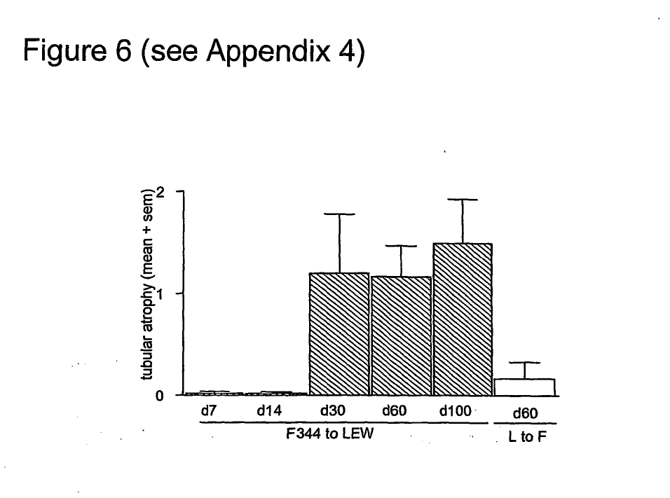

FIG. 7 shows telomere restriction fragment (TRF) length as measured with the TeloREAD assay in normal F344 and LEW kidneys or in F344 and LEW allografts on day 60 (A). Telomere length in F344 to LEW renal allografts in time after transplantation (B). Values are expressed as mean of 3 samples+standard error of the mean (sem).

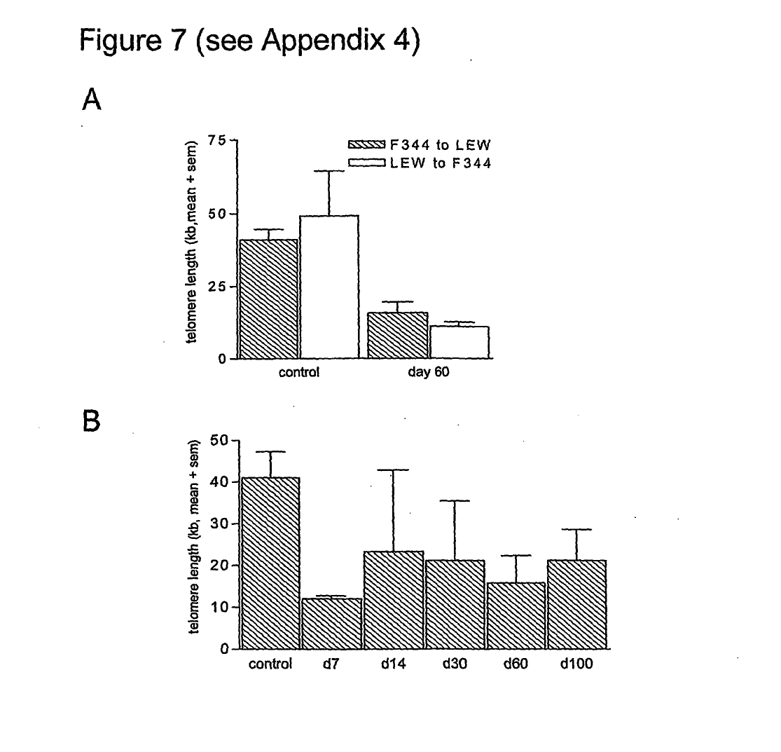

FIG. 8 shows : A: Western blot analysis of p21 and actin expression in lysates of normal and F344 to LEW renal allografts (day 7). Samples are representative for 3 samples in each group. B: Densitometry of p21 Western blots of F344 and LEW renal allografts at various time points after transplantation, corrected for actin content of the samples. Data are expressed as mean+standard error of the mean (sem). (n=3 samples for all groups).

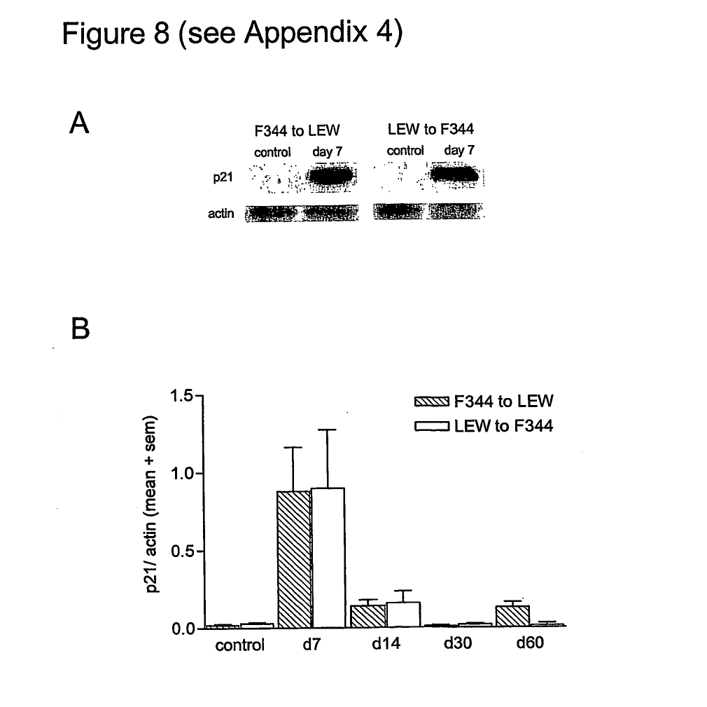

FIG. 9 shows: A: Immunohistochemical staining of nuclear p16 protein in normal F344 (left) and F344 to LEW allograft at day 60 (middle). No nuclear staining is observed in the absence of the primary antibody (right). (400×) B: Densitometry of total p16 staining on Western blot of F344 and LEW renal allografts on various time points after transplantation, corrected for actin content of the samples. Data are expressed as mean+standard error of the mean (sem). (n=3 samples for all groups).

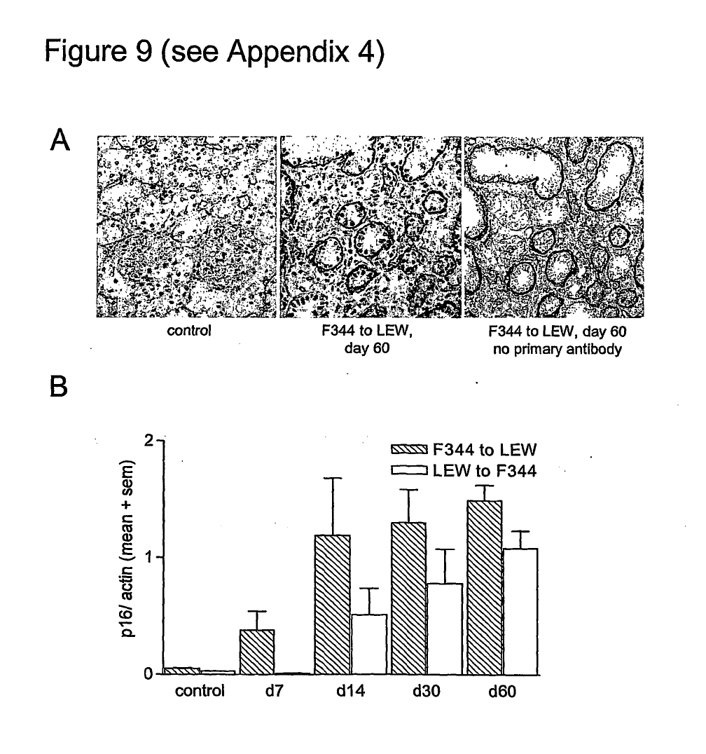

FIG. 10 shows: A: Senescence associated β-galactosidase staining (SA β-gal) (pH 6.0) in frozen sections of normal F344 kidney, F344 to LEW renal allograft at day 60 and a LEW to F344 renal allograft at day 60. Biopsies are representative for 6 biopsies in each group. (×250) B: Quantification of SA β-gal staining using Zeiss ks400 analysis for normal F344 kidneys, F344 and LEW renal allografts on day 60 post transplant. Data are expressed as mean+standard error of the mean (sem) (n=6 for all groups). C: Quantification of SA β-gal staining using Zeiss ks400 analysis. F344 to LEW day 7 (n=3), day 14 (n=3), day 30 (n=3), day 60 (n=6) and day 100 (n=3), data expressed as mean+standard error of the mean (sem).

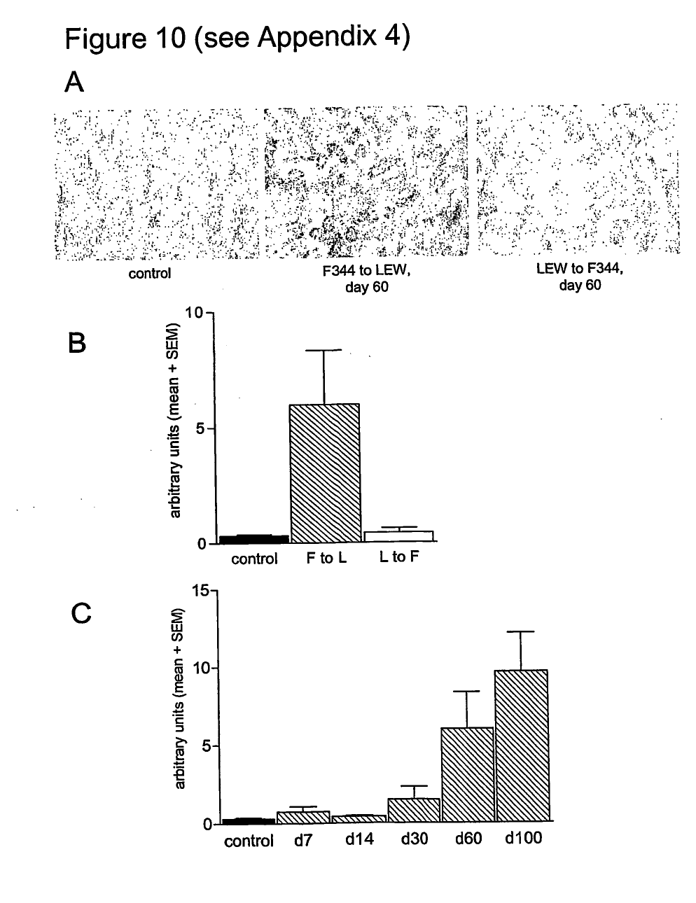

FIG. 11 shows: A: SA β-gal staining of F344 to LEW allograft (left) and F344 to F344 syngraft (right) on day 60 post transplant. (×250) B: Telomere restriction fragment (TRF) length of normal F344 and LEW kidneys (n=3) compared with F344 to F344 (day 14 and 60, n=3) and LEW to LEW syngrafts (day 14, n=3), expressed as mean+standard error of the mean (sem).

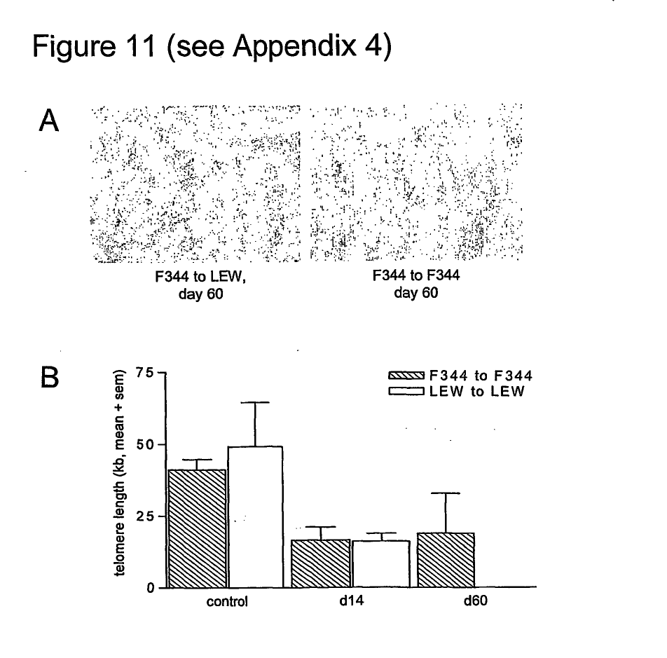

FIG. 12 shows telomere restriction fragment length of F344 kidneys after 45 minutes of ischaemia (n=3) and 45 minutes of ischaemia followed by 2 hours of reperfusion (n=3), expressed as mean+standard error of the mean (sem).

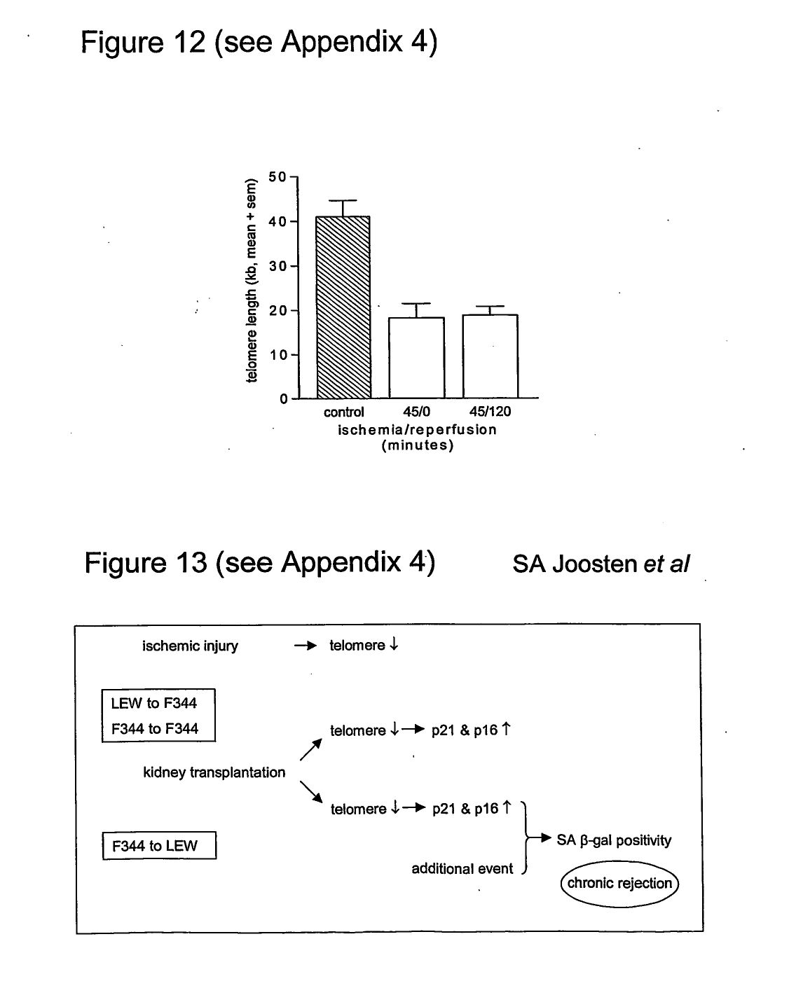

FIG. 13 shows a schematic representation of events as observed in the F344 to LEW and LEW to F344 renal allografts. Transplantation results in shortening of telomeres in all combinations investigated, this results in activation of p21 and p16. However, only in F344 to LEW renal allografts SA β-gal staining is observed, this suggests that an additional event is required to induce cellular senescence.

The present application includes a number of Appendices, which represent various publications by the present inventor and collaborators which are currently in press. The appendices are not essential to the understanding or implementation of the invention, but are included to provide a more detailed background understanding of the subject-matter of the invention. Details of the four appendices are given below. FIG. 4 is taken from Appendix 2, FIG. 5 from Appendix 3, and FIGS. 6 to 13 are taken from Appendix 4.

Appendix 1 is a paper entitled “Senescence and Transplantation” by Nolan C E, Wright S M and Shiels P G. Transplant Topics (2003), in press.

Appendix 2 is a paper entitled “Dolly, no longer the exception; telomeres and implications for transplantation”. Shiels P G and A G Jardine. Cloning and Stem Cells (2003), in press.

Appendix 3 is a paper entitled “Cellular senescence, organ ageing and transplantation”. Shiels P G Nolan C E and Sklavouou E. Am J Transpl. (2003), in press.

Appendix 4 is a paper entitled “Telomere shortening and cellular senescence in a model of chronic renal allograft rejection”, by Joosten S A, van Ham V, Nolan C E, Borrias M C, Jardine A G, Shiels P G, van Kooten C, and Paul L C; Am J Path (2003), in press.

Before discussing the various Figures, for a better understanding of the invention, some further background detail will be given on the senescence of cells and the action of the relevant proteins.

Telomere Instability

Telomeres are specialised structures found at the end of eukaryotic chromosomes consisting of simple repetitive DNA, which in mammals comprises the sequence (TTAGGG)ₙ. Without specialised structures to cap telomeres and to replicate them during cell division, the telomeres gradually shorten over time. It is generally believed that shortening of telomere length through repeated cell divisions over time contributes to the ageing and senescence of somatic cells (reviewed in Shiels 1999).

The telomere capping function appears to be mediated by a combination of a unique tertiary structure and specific telomere binding proteins. Electron microscopy has shown that the mammalian telomere takes the form of a loop, termed the t-loop, created by the telomere DNA folding back on itself to form a lariat whose leading end is the telomeric 3′ G strand overhang. This is envisaged as invading adjacent duplex telomeric repeats, thus creating a displacement loop (D-loop). Duplex DNA binding proteins are proposed to bind along the telomeric repeats of the t-loop, while a specialised DNA binding protein stabilises the D-loop lariat junction (Griffith et al., 1999)

Recently a protein capable of binding the single stranded G-rich extension found at the ends of all telomeres has been identified (Baumann and Cech, 2001). This protein has been termed POT1 (Protection of Telomeres) and appears conserved in eukaryotes as diverse as yeast and humans. Deletion of the fission yeast pot1 gene produces immediate chromosome instability and rapid loss of telomeric DNA. The capping of telomeres thus appears to be crucial for avoiding chromosome instability and descent into cell death. In telomerase negative human somatic cells, telomeres may shorten with each cell division, finally reaching a critical level. However, cell death will not be triggered unless the telomere loses its capping proteins and becomes unstable. The state of the telomere (capped or uncapped) may also determine the degree of shortening which occurs with each cell division. Therefore telomere binding proteins may contribute directly to the internal mechanism which determines when the cell enters senescence.

Oxidative Damage

Free radicals (ROS) are known to promote DNA damage and hence to contribute to the accumulation of damage which can promote cellular senescence. Oxidative metabolism is further known to promote production of ROS, and so promote senescence. Indeed, it has further been found that slowing metabolism of organisms, for example by restricting food supply, prolongs life span to some extent. A number of observations provide an insight into how ROS may speed the entry of a cell into senescence.

Firstly, increased oxygen tension accelerates telomere erosion in replicating human fibroblasts in vitro (von Zglinicki and Schewe, 1995). Secondly, it has been observed that telomeres accumulated a significantly higher frequency of single-stranded overhangs, gaps and single-stranded breaks than the bulk of the genome when human fibroblasts were subjected to chronic oxidative stress (Peterson et al, 1998). Conversely, when human vascular endothelial cells were grown with an oxidation-resistant type of ascorbic acid, they showed a decrease in rate of telomere shortening by 52-62%, and an extension of cellular life span when compared to controls (Furumoto et al, 1998).

One possible explanation for this is that the G rich base repeat of telomeres is particularly susceptible to damage from ROS. This has been observed in lower eukaryotes where disruption of telomere structure occurs as a result of such damage. It is also possible that DNA damage to genomic DNA precipitated by ROS results in vital telomere binding proteins being recruited from the telomeres to the site of genomic DNA as part of the repair mechanism. This would leave the telomeres exposed and uncapped, and thus more susceptible to instability.

Human in vivo evidence for an acceleration of telomere shortening in conditions of oxidative stress can be found be examination of telomeres of patients with respiratory chain disorders. These conditions are associated with an increased production of ROS due to faulty mitochondrial respiration, and telomeres in these patients were on average 1.5 kb shorter than those of controls (Oexle and Zwirner, 1997).

Molecular mechanisms underlying pathogenesis of CAN The above observations are consistent with a hypothesis in which oxidative damage can lead to a senescent phenotype, indicating that such changes underlie the development of CAN. Such damage is particularly relevant to transplantation and grafting, where the donor tissue may have been subject to ischaemic and hypoxic stress, with consequent changes in metabolism and production of ROS. Further relevant observations include:
The presence of senescent features such as telomere erosion in cloned sheep and cattle (Shiels et al. 1999a, b; Miyashita et al 2002) despite the animal appearing physiologically normal is in keeping with a model of senescence based on oxidative damage. These observations and their relevance to transplantation are discussed more fully in Appendix 2.
Age related changes have been observed in mitochondrial DNA sequence and conformation (Kopsidas et al, 2000) along with an altered DNA end binding capacity, significantly, mediated through XRCC5 (Coffey and Campbell 1999)
Mitochondrial xanthine oxidoreductase, a known cause of ischaemic reperfusion damage shows altered expression with age, resulting in elevated mitochondrial oxidative stress levels (Chung et al, 1999).
In vivo murine gene expression analysis and in vitro models of interstitial fibrosis indicate specific perturbation of the genes for telomere binding proteins, anti oxidant enzymes, and ribosomal components, in common with senescing cells.
Mutations altering the function of telomere binding proteins can result in progeria (Myung et al, 2000).
Specific age related changes in the expression of genes involved in the formation of the cytoskeleton and the extracellular matrix are also observed in kidneys undergoing CAN (Linskens et al, 1995).

At a molecular level, much of the damage of a prolonged ischaemic time will result directly from the ROS produced when hypoxic cells are reoxygenated. An increase in chronic rejection of transplants has been observed to correlate to prolonged ischaemic times.

ROS can accelerate cellular senescence directly by the mechanisms described above. Therefore the degree of senescence, both cellular and physiological (see Appendix 3) already present within the donor tissue will become important, representing the degree of reserve that the cells have. Those cells that are nearer the end of their life span (for example, with shorter telomeres) will be more susceptible to damage from ROS and will enter senescence faster. Organs or tissues exhibiting physiological senescence will also be more susceptible to ischaemia/reperfusion injury and thus prone to accelerated physiological senescence post transplant. This is more fully discussed in Appendix 1.

Without wishing to be bound by theory, the present inventors believe that oxidative stresses affecting transplant rejection result in changes in the production of ROS, and consequently the expression of telomere binding proteins. In particular, expression changes in G22P1, XRCC5, hPOT1, and SIRT2 have been investigated. These proteins are involved in the maintenance of telomere structure, DNA repair, and silencing of subtelomeric regions.

G22P1 and XRCC5 monomers form part of a complex that was originally identified as a major autoantigen in patients with autoimmune diseases such as scleroderma. It is known to bind the ends of double-stranded DNA with high affinity and to be involved in repair of DNA by NHEJ. They are also known to be specifically co-located at the telomeres with SIRT2 and Rap1, and to play an important role in telomere maintenance in yeast. These products are known to interact with the Werner helicase protein, and are also responsible for the silencing of genes adjacent the telomere (Nugent et al 1998) and are found along with SIRT2 along this silenced region next to the telomere. In yeast, loss of the analogous heterodimer, Ku, from the telomere promotes loss of SIRT2, for example when Ku is recruited to repair a double stranded DNA break.

It is believed that the expression of telomere binding proteins is diagnostic for CAN and graft suitability. The present inventors suggest that DNA damage accrued pre-transplant results in substantial upregulation of G22P1 and XRCC5 expression, with concomitant telomere destabilisation as their respective gene products are recruited to DNA breaks elsewhere in the chromosome. Increase in hPOT1 expression occurs in an attempt to offset the destabilisation. Further, enhancement of expression of these proteins in the pre-transplant tissue would be expected to reduce the risk of ROS damage to the cell, and hence lessen the risk of subsequent tissue rejection. Post-transplant intervention may also be possible to lessen the risk of subsequent damage and hence rejection.

Materials and Methods

RNA Isolation. Small frozen biopsy (10-50 mg) samples from human allografts were used to prepare RNA using Trizol (Sigma-Aldrich Co. Ltd, Poole, UK) following manufacturer's recommendations.

Samples were homogenised in Trizol using a PowerGen 125 Tissue Homogenizer (Fisher Scientific) using a test tube pestle. The homogenisation was performed until a homogeneous solution was obtained and very few visible tissue pieces were present. Samples were then incubated at room temperature for 5-10 minutes and the homogenate transferred to a centrifuge tube, and centrifuged at 12,000g (10,000 RPM on SS34) for 5-10 minutes at 4° C.

The upper layer was removed using a Pasteur pipette and 0.2 ml chloroform added per 1 ml Trizol reagent used initially. The tube was shaken vigorously for 15-30 seconds, then incubated at room temperature for 5 minutes. Samples were then spun at 12,000 g for 15 minutes at 4° C. The upper aqueous phase, which contains the total RNA, was removed and placed in a new centrifuge tube.

The RNA was precipitated by adding 0.5 ml isopropyl alcohol per 1 ml Trizol reagent used, and then incubated at room temperature for 10 minutes, followed by centrifugation at 12,000 g for 10 minutes at 4° C. The supernatant was then decanted and the pellet washed once using 70% ethanol and spun at 7,500 g (7,500 RPM in SS34) for 5 minutes at 4° C. The supernatant was again decanted and the pellet air dried briefly. The RNA pellet was then resuspended in 200 μl DEPC water, and quantified by spectrophotometry.

Slot blotting. The slot blot apparatus was assembled using Hybond N+ nylon membrane (Amersham, UK) as the solid support in accordance with the manufacturer's instructions, particular care being taken that no leakage occurred between wells before loading the RNA samples. This was tested using Orange G in sterile water as a marker dye. The wells were rinsed twice with 10×SSC and fluid sucked through the manifold under vacuum. The vacuum was then turned off, and a further 50 ml of 10×SSC added to each well. Two volumes of RNA loading buffer was added to each 5 μg sample of RNA, which were then denatured at 70° C. for 10 minutes, and chilled on ice. RNA samples were then loaded into the wells already containing 10×SSC and aspirated through under vacuum. The wells were then rinsed twice with 200 μl of 10×SSC and this drawn through under vacuum. The resulting slot blot was then rinsed in 2×SSC and briefly air dried, then the RNA UV cross linked in place and stored at room temperature until required for hybridisation.

Hybridisation. This was performed at 65° C. in 6×SSC overnight with the respective probes labelled with α-³²P. Subsequent washing post-hybridisation was to a stringency of 0.2×SSC at 65° C. The blot was then subjected to analysis using a Molecular Dynamics PhosphorImager. Statistical evaluation of signals was conducted using Microsoft Excel.

Generation of hybridisation probes. Probes were developed by RT-PCR of human mesangial cell RNA, using primers designed to amplify parts of the mRNA sequence of the gene of interest. Primers for hPOT1 were selected according to published data (Baumann and Cech, 2001). Primer sets for SIRT2, G22P1 and XRCC5 were developed from the respective GenBank sequence records using a specific algorithm provided by Vector NTI suite (Informax). Primer sequences are as follows:

| | |
|---|---|
| 1. XRCC5 | |
| Primer Sense: | CTG AGG ACC GGC AAC ATG GT |
| Primer Antisense: | CCA TCT TCC TTG CCA AGT GA |
| 2. G22P1 | |
| Primer Sense: | AAC ACG TCT CTT GCC AGG TC |
| Primer Antisense: | CAC AAG TCT GGT GGT GGT GT |
| 3. hPOT1 | |
| Primer Sense: | GGA AGT GCA GCA GGG TTC AG |
| Primer Antisense: | GGT TCT GCT TCC CTT TCA GT |
| 4. SIRT2 | |
| Primer Sense: | CCG CTA AGC TGG ATG AAA GA |
| Primer Antisense: | GAC TGG GCA TCT ATG CTG GC |

The level of gene expression was then measured by in-situ hybridisation with the appropriate amplicons.

HTEC culture. Primary HTEC cultures were grown at 37° C. 5% CO₂ in DMEM/Glutamax-1/+ pyridoxine medium (Life technologies) plus 20pg ml⁻¹ Penstrep. Cells were stained for SA β Gal activity as described in Joosten et al 2003.

Experiment 1

Consent was obtained to take a small biopsy (10-50 mg) of renal tissue from the allografts at transplantation or nephrectomy. RNA was extracted from this biopsy and 5 μg transferred by means of a slot blot onto Hybond N+ membrane. Probes were developed by RT-PCR of human mesangial cell RNA using primers as described above. The level of gene expression was then measured by in situ hybridisation with the appropriate α-³²P labelled probe. Phospho-images of the hybridised blots were then developed and analysed using ImageQuant technology.

A total of 4 samples from allografts with CAN, 4 from T₀ cadaveric donor biopsies, and 8 from T₀ living donor biopsies was analysed on multiple Northern slot blots. Comparative expression data from a representative blot is illustrated in FIG. 1. This indicates that there was an increase in the expression of G22P1 and XRCC5 in the CAN samples when compared to T₀ living and cadaveric samples. Statistical analysis using a split-plot analysis of variance was undertaken to compare expression for each gene in a) T₀ living and T₀ cadaveric samples; and b) T₀ living and CAN samples, with comparison being made between the three biopsy classes for the four genes tested. The raw data from the analysis is shown in FIG. 2.

Overall kidney type differences were assessed by comparison with the residual variation between samples and are significant at p<0.01. Gene differences were assessed by comparison with the residual variation within samples and are significant at p<0.001. Overall differences between kidney types with genes were assessed by comparison with a combination of between and within sample variation, and are significant at p<0.05. G22P1, XRCC5, and hPOT1 analysed within the respective groups showed significant differences in expression (at p<0.05). Surprisingly, there was also a significant increase in expression of SIRT2 in T₀ living samples when compared to T₀ cadaveric, despite no difference being observed between T₀ living and CAN samples (p<0.05).

The increased expression of G22P1, XRCC5, and hPOT1 in CAN biopsies indicates that cellular senescence is a contributory factor in the pathogenesis of CAN. The reduction of SIRT2 expression in To cadaveric samples when compared to T₀ living samples is interesting, and may reflect the mechanism by which oxidative damage promotes cellular senescence and therefore CAN, as this was not observed for the other three senescence associated genes. The observed differences in expression data do not appear to be correlated with the age of the donor organ, but rather are a specific reflection of senescence associated processes.

These results indicate that the G22P1, XRCC5, and hPOT1 genes are early markers for the development of CAN, and suggest a method for the screening of donor tissues prior to transplantation. For example, if features of cellular senescence, such as increased expression of G22P1 and XRCC5, are found in donor kidneys, it might be assumed that these grafts are more likely to develop CAN than those with low levels of expression of these genes. Routine biopsies that indicated a sharp increase in the expression of G22P1 or XRCC5 and therefore the imminent development of CAN would contribute to the more precise management of the transplanted kidney, or to the suppression of the development of CAN. Further, the results of analysis of SIRT2 expression indicate that a decrease in SIRT2 expression in cadaveric samples may be prognostic for the future utility of cadaveric tissues. Enhancement of expression of all four genes may be expected to provide additional protein for cell and DNA maintenance and repair, and so prolong the effective lifespan of transplanted organs and reduce the risk of rejection.

Experiment 2

The present invention has been exemplified in a rat model of chronic rejection. This is more fully described, with materials and methods, in Appendix 4 (Joosten et al 2003), but is summarised below.

In a series of pilot experiments, accelerated senescence has been demonstrated in an experimental model of chromic kidney rejection in the rat. In this model, rejecting allografts show senescence associated β galactosidase (SA β gal) staining, elevated p16 and p21 expression and massive telomere erosion (see Appendix 4 for full details). These data are highly significant, in that they demonstrate rapid telomere loss due to as little as 45 minutes ischaemia. Critically, the degree of loss is too great to be accountable by replicative senescence and is strictly non-immune in origin. This observation is consistent with apoptosis within the allograft engendering physiological senescence. Specific telomere erosion is an early event in apoptosis that precedes caspase activation (Ramirez et al 2002). Significantly, rejecting kidneys only show SA β gal staining in tubular epithelium. This is discussed more fully in Appendices 2 and 3 and is summarised below.

The apparent specific susceptibility of epithelial cells in rejecting kidneys to senesce is consistent with observations in animals derived by nuclear transplantation (cloning). Animals derived from epithelial cell donor nuclei all appear to have shortened telomeres, indicative of an inability to fully rectify the effects of senescence in these nuclei (Shiels et al 1999a, b; Shiels 1999; Miyashita et al 2002). This apparent susceptibility of epithelial cells to stress induced senescence is further supported by the observation that the transporter for uric acid (URAT1), the main anti-oxidant in humans, is found solely on tubular epithelial cells in the kidney. Significantly, URAT1 mutations correlate with the development of idiopathic hypouricaemia, a fatal condition characterised by exercise induced production of reactive oxygen species (ROS) in the kidney (Enamoto et al 2002).

Experiment 3 In vitro Model for Human Renal Senescence

Building upon the rodent observations, the inventor has established a primary human tubular epithelial cell (HTEC) in vitro system to model and evaluate premature human renal senescence. Data from this system indicates that HTECs senescence differently in the presence of individual calcineurin inhibitors, as judged on the basis of SA β Gal staining. This is shown in FIG. 3, which illustrates SA β Gal staining at pH6 in primary tubular epithelial cultures as a measure of senescence.

Cultures were grown ±100 ng/ml cyclosporin (cyc) or 10 ng/ml tacrolimus (FK) and evaluated with or without oxidising 75 μM H₂O₂ treatment. Cyclosporin appears to exacerbate oxidative damage in this system, which has implications for conventional immunotherapy.

One such inhibitor, tacrolimus, appears to protect against the effects of oxidant stress when the cells are challenged by treatment with the oxidant hydrogen peroxide (H₂O₂). This has profound implications for transplant outcome and current clinical practice. This data is consistent with recent data reporting that calcineurin inhibitors are cytoprotective and prevent apoptosis (Shirane et al 2003).

References

Baumann P, Cech T R (2001). Pot1, the putative telomere end-binding protein in fission yeast and humans. Science. 292:1171-5.
Chung H Y, Song S H, Kim H J, Ikeno Y, Yu B P. (1999). Modulation of renal xanthine oxidoreductase in ageing: gene expression and reactive oxygen species generation. J Nutr Health Ageing. 3:19-23.
Coffey G and Campbell C (1999) An alternate form of Ku 80 is required for DNA end-binding activity in mammalian cells. Nuc. Acids Res 28: 3793-3800.
Enomoto A, Kimura H, Chairoungdua A, Shigeta Y, Jutabha Pet al (2002) Molecular identification of a renal urate anion exchanger that regulates blood urate levels. Nature. 417:447-452
Furumoto K, Inoue E, Nagao N, Hiyama E, Miwa N (1998). Age-dependent telomere shortening is slowed down by enrichment of intracellular vitamin C via suppression of oxidative stress. Life Sci. 63:935-48.
Griffith J D, Comeau L, Rosenfield S, Stansel R M, Bianchi A, Moss H, de Lange T (1999). Mammalian telomeres end in a large duplex loop. Cell. 14;97:503-14.
Halloran P, Melk A, Barth C (1999) Rethinking chronic allograft nephropathy: the concept of accelerated senescence. Am Soc Nephrol, 10:167-181
Kopsidas G, Kovalenko S A, Heffernan D R, Yarovaya N, Kramarova L, Stojanovski D, Borg J, Islam M M, Caragounis A, Linnane A W. (2000). Tissue mitochondrial DNA changes. A stochastic system. Ann N Y Acad Sci. 908:226-43.
Linskens M H, Feng J, Andrews W H, Enlow B E, Saati S M, Tonkin L A, Funk W D, Villeponteau B. (1995) Cataloguing altered gene expression in young and senescent cells using enhanced differential display. Nucleic Acids Res. 23:3244-51.
Miyashita N, Shiga K, Yonai M, Kaneyama K, Kobayashi S, et al (2002) Remarkable differences in telomere lengths among cloned cattle derived from different cell types. Biol Reprod. 66:1649-1655.
Myung K, Datta A, Chen C and Kolodner R. (2000) SGS1, the Saccharomyces cerevisiae homologue of BLM and WRN, suppresses genome instability and homeologous recombination. Nature Genet. 27: 113-116.
Nugent C I, Bosco G, Ross L O, Evans S K, Salinger A P, Moore J K, Haber J E, Lundblad V (1998). Telomere maintenance is dependent on activities required for end repair of double-strand breaks. Curr Biol. 8:657-60.
Oexle K, Zwirner A (1997). Advanced telomere shortening in respiratory chain disorders. Hum Mol Genet. 6:905-8.
Paul L C (1999). Chronic allograft nephropathy: An update. Kidney Int. 56:783-93.
Petersen S, Saretzki G, von Zglinicki T (1998). Preferential accumulation of single-stranded regions in telomeres of human fibroblasts. Exp Cell Res. 239(1):152-60.
Ramirez R, Carracedo J, Jimenez R, et al (2002) Massive telomere loss is an early event of DNA damage-induced apoptosis. J Biol Chem. 2002 Oct. 29 E Pub ahead of print.
Shiels P G (1999) Somatic cell nuclear transfer as a tool for investigating ageing processes in Mammals Gene Ther. Mol Biol 4: 11-22.
Shiels P G, Kind A J, Campbell K H, Waddington D, Wilmut I, Colman A, Schnieke A E (1999a) Analysis of telomere lengths in cloned sheep. Nature 399: 316-317.
Shiels P G, Kind A J, Campbell K H, Waddington D, Wilmut I, Colman A, Schnieke A E (1999b) Analysis of telomere length in Dolly, a sheep derived by nuclear transfer. Cloning 1(2):1-7.
Shirane M and Nakayama K I (2003) Inherent calcineurin inhibitor fkbp38 targets bcl-2 to mitochondria and inhibits apoptosis. Nat Cell Biol 5: 28-37
von Zglinicki T, Schewe C (1995). Mitochondrial water loss and ageing of cells. Cell Biochem Funct. 13:181-7.
   Appendix 1
   Senescence and Transplantation

The demand for donor organs continues to increase, and is not met by the current availability of cadaveric organs. Alternative strategies such as xenotransplantation¹⁻³ and use of “marginal” donors provide potential solutions in the longer term, as does a better understanding of what causes a transplanted organ to fail. A further option is stem cell therapy, which while potentially very promising, is still in its infancy.

It is becoming more apparent that the mechanisms of ageing, at the level of the organism, organ and the cell, hold important lessons for disease susceptibility and disease state progression. This is particularly relevant to late (chronic) transplant rejection. It has been proposed that oxidative damage at the time of transplantation sets in motion a series of events that results in accelerated senescence within the graft⁴⁻⁵ leaving it less able to cope with further stress. Consequently, the graft is more vulnerable to immune and non-immune mediated injury and is predisposed to disease. This review focuses on the current understanding of senescence and offers an appraisal of how this may contribute significantly to the success of transplanted organs.

Senescence

Senescence can be resolved into three component parts:
Chronological ageing—essentially the time elapsed since birth. This is characterised by declining organ mass, integrity and function resulting from the cumulative burden of environmental and metabolic insults.
Cellular senescence—ageing at a cellular level. This is characterised by growth arrest and loss of replicative potential. This is due principally to oxidative damage to DNA and the oxidation of proteins and lipids.
Physiological senescence—a decline in physiological capacity resulting from chronological ageing and cellular senescence. Critically, the degree of physiological senescence will be dependent upon the numbers and location of cells lost through insult, apoptosis or cellular senescence.

It is worth noting that physiological and cellular senescence are distinct entities, though they overlap extensively.

In mammals, cellular senescence is considered an evolutionary mechanism to counter neoplasia⁶. The growth arrest characteristic of cellular senescence, is typified by the erosion of telomeres and the expression of specific senescence associated genes (SAGs), including those involved in cell cycle control, anti-oxidant defences, telomere stabilisation, maintenance of ribosomal DNA integrity, cytoskeleton and extracellular matrix formation (Table 1).

Telomeres and Stress Responses

Telomeres are specialised nucleoprotein complexes found at the end of eukaryotic chromosomes. In mammals, they comprise stretches of the simple DNA repeat (TTAGGG)ₙ⁷ bound with a complex and dynamic array of proteins that function to cap the free DNA end, sense and signal DNA damage and facilitate DNA repair. An inability to respond to, or repair damage properly, can result in accelerated ageing. Progeric conditions, such as Werners' syndrome, Blooms' syndrome and Hutchinson-Guilfords' disease all result from the dysfunction of telomeric proteins.

Critically, telomeres facilitate the complete replication of chromosome ends⁸. Somatic cell telomeres in man shorten during replicative ageing through incomplete replication of chromosome ends and the loss of telomeric DNA. This may provide a mitotic clock that eventually signals cell death. In germ cells and stem cells, this erosion is countered by the action of the enzyme telomerase. Mice deficient in telomerase activity progressively lose telomeric DNA and display premature ageing phenotypes⁹. Reintroduction of telomerase not only lengthens the telomeres in these transgenics, but can also prevent premature ageing¹⁰. Telomere shortening has been causally implicated in human cellular senescence, disease and by general implication, the physiological ageing process in higher animals¹¹⁻¹³.

From Cell to Organism

Extrapolating observations on cellular senescence to the level of the organism is not always straightforward. When a cell is subject to oxidant stress, a series of integrated responses ensues, linking a functional trinity, comprising the mitochondrion, the telomere and the ribosome (see FIG. 4). If the level of damage is too great the cell will undergo apoptosis. If the level is sub lethal it will signal repair and senescence. This will have a direct effect on organ physiology following the stress, and will be discussed below in the context of transplantation. The advent of nuclear transplantation (cloning) has allowed the integration of in vitro observations with those on living organisms. Significantly, it has provided some novel insights into how different cell types handle stress and how this is might be reflected in the physiology of cloned animals. These data have substantial implications for transplantation.

The cell type of the nucleus to be cloned appears to be critical in determining if the effects of cellular senescence are negated in resultant clones. Clones derived from epithelial cell donor nuclei all appear to have shortened telomeres¹⁴⁻¹⁶, in contrast to those derived from fibroblasts or muscle cells¹⁶⁻²⁰. This indicates that different cell types may have different capacities to both withstand and repair the effects of oxidative insult. In turn, this would influence the degree of physiological senescence in the organs of the cloned animal. If critically short telomeres were reached in the animals' life time then the effects of such senescence might manifest in early onset of diseases of ageing. Observations in cloned sheep and cattle indicate that replicative senescence alone is insufficient to precipitate such a state of affairs, as the rate of telomere erosion in vivo is too slow and the animals appear to have sufficient telomeric DNA to prevent a crisis being reached in their lifetime. This situation may not hold true for transplanted cells or organs as will be discussed below.

Chronic Allograft Nephropathy

Chronic allograft nephropathy (CAN), is characterised by progressive impairment of renal allograft function. It is the main cause of renal allograft failure in the first post-transplant decade²¹. Its pathogenesis is undetermined, and as a consequence is difficult to prevent or treat, although, histologically it exhibits features in common with chronological ageing. It is also associated with increased donor age, suggesting a role for cellular senescence. This is supported by common gene expression profiles in senescent cells and rejecting kidneys²². Consequently, accelerated senescence in organ transplantation has been proposed to underlie the progressive changes seen in CAN⁴. This hypothesis can be addressed by a number of key questions.

Why does Accelerated Senescence Arise in Renal Allografts?

Ischaemia and reperfusion injury provide a ‘double hit’ that can have profound consequences for graft function. The impact of ischaemia/reperfusion (I/R) injury and subsequent oxidative damage to the allograft has previously been underestimated. Animal studies have confirmed elevated levels of reactive oxygen species (ROS)-mediated lipid peroxidation in tissues of acutely rejected rat cardiac allograft²³ and increased liberation of free radicals has been shown in the serum of transplant recipients post transplant²⁴. Parenthetically, apoptosis and inflammation are recognised consequences of I/R injury and free radical mediated damage²⁴.

However, if these processes were of importance in graft function, we would expect to see a reduction in I/R injury and improved graft outcome following anti-oxidant therapy. In fact, a significantly improved 1 and 4 year graft survival rate and reduction in first acute rejection episodes after cadaveric renal transplantation has been reported in patients treated with superoxide dismutase (SOD), a scavenger of ROS²⁶. This result has not always been reproducible²⁷ and may reflect the means and timing of SOD administration, dosage used, short half-life of SOD, lack of suitable targeting (e.g. to the mitochondrion), and the fact that SOD itself leads to hydrogen peroxide formation and may therefore not be the best anti-oxidant in this context. More recently, focus has shifted to other anti-oxidants. Of considerable interest is the finding that induction of heme oxygenase-1 protects against tissue injury²⁸⁻²⁹ and the emerging evidence of the protective effects of ischaemic pre-conditioning in I/R injury, and the possible role for immunosuppressants in this setting.

It is not unreasonable to suggest that oxidative damage to essential cellular macromolecules at the time of transplantation results in cell damage, which may signal the cell down apoptotic pathways if the damage is too great, or alternatively, into repair pathways which may ultimately lead to cellular senescence. A consequence would be an organ which is susceptible to further injury, and is more likely to display hallmarks of chronic dysfunction. A clearer understanding of the role of senescence in transplantation is therefore essential for understanding graft failure, identifying novel therapeutic targets, and modulating existing immunotherapies.

How Might these Effects Manifest within the Graft?

Ischaemic damage and reperfusion injury will result in massive telomere destabilisation and altered expression of SAGs. Specific telomere erosion is an early event in apoptosis and precedes activation of the caspase cascade in the mitochondrion. Our laboratory, in collaboration with the group of Leen Paul in Leiden, have observed massive telomere loss in a rat model of renal allograft rejection with as little as 45 minutes ischaemic time. Significantly, this loss is accompanied by subsequent elevation in the expression of p21 and p16, classic molecular markers of senescence, following reperfusion³⁰. These observations are consistent with the development of accelerated senescence in these allografts. Critically, senescence associated beta galactosidase expression (SA beta Gal), a classical cytological marker of senescence associated oxidant stress, is limited solely to rejecting grafts. These data are paralleled by changes in the expression of telomere binding proteins (table 1) linked to signalling and repair of DNA damage³¹. We have also observed significant changes in SAG expression with CAN in man.

These observations are supported by data from a rat model of ischaemia and reperfusion injury³² which demonstrate that the extent of tissue senescence, as determined by amount of SA beta Gal positively staining cells, correlates inversely with recovery of function after I/R injury.

One significant feature of the chronic renal allograft rejection model, is that the cellular senescence, as determined by SA beta Gal staining, is limited to tubular epithelium. This has profound implications for the physiological impact of transplant related oxidant stress. The apparent susceptibility of this cell type to oxidative injury is in keeping with two key observations. (i) As discussed previously, animals cloned from epithelial cell show advanced telomere erosion and do not appear to be able to fully repair senescence associated oxidative damage¹⁴⁻¹⁶. (ii) The Uric acid transporter (URAT1) is found solely on tubular epithelium in the kidney. Uric acid is the primary anti-oxidant in the-human blood. Mutations in URAT1 are responsible for idiopathic renal hypouricaemia, a condition typified by exercise induced acute renal failure and ROS induced tubule cell death, which is consistent with the above hypothesis.

Implications and Intervention

Tackling senescence, at the physiological and cellular levels, has implications for both allo- and xeno-cellular and solid organ transplants. I/R injury will not discriminate between a pig or a human organ and the outcome will be the same. In a xenotransplant setting this may even be exacerbated, if the xenograft source is an animal derived by nuclear transplantation. In this instance the endogenous telomere lengths of the animal are critical in order that critical telomere lengths are not reached in an organ post transplant. A graft from an animal with telomere lengths shorter than age matched controls, might prove to be compromised once subject to I/R injury. In an allo-transplant setting this would imply that grafts from older donors would function less well post transplant, as might those from cadavers. This again is consistent with clinical observations.

The situation for cellular transplants is more complicated, in that cell populations differentiated in culture already show increased levels of oxidative damage and accelerated senescence. Stem cell therapies using in vivo differentiation strategies will potentially avert this, though control of the differentiation of such transplants in vivo is still in its infancy. Of more concern is the recent observation that simply using telomerase to combat telomere erosion may not be sufficient for differentiated cell transplants. If the telomerase activity is insufficient, the cells may bypass senescence, but retain an unstable genome, which may then predispose to neoplasia³³.

Therapeutic intervention in this area appears likely. The simplest approach being to combat I/R using pre-transplant administration of suitable anti-oxidants. Targeting of the anti-oxidant to the mitochondrion may prove to be beneficial, given its relationship with the telomere-nucleoprotein complex. Anti-apoptotic agents will also prove crucial in this setting. Gene therapy strategies may also be feasible, targeting tubular epithelium to assist in reducing oxidant stress and enhancing DNA repair capacity, or tackling areas of focal damage post transplant.

One final advantage of an understanding of how senescence relates to transplantation, is that it will provide a molecular standard for graft prognosis and disease progression, free from observer bias.

References

1. Dorling A, Riesbeck K, Warrens A, et al (1997). Lancet 349:867-871
2. Dorling A, Lechler R I. (1998). Xenotransplantation 5: 234-245.
3. Ayres D, Colman A, Dai Y, et al (2001). Graft 4: 80-82
4. Halloran P F, Melk A, Barth C (1999). J Am Soc Nephrol 10; 167-181
5. Shiels P G and Jardine A G (2002). Cloning and Stem cells submitted.
6. Shiels P G (1999a). Gene Therapy Mol Biol 4: 11-22
7. Moyzis R K, Buckingham J M, Cram L S, et al (1988). Proc Nat Acad Sci USA 85: 6622-6626
8. Olovnikov A M (1973). J Theor Biol 41: 181-190
9. Blasco M A, Lee H-W, Hande P et al (1997). Cell 91: 25-34
10. Samper E, Flores J M and Blasco M A (2001). EMBO rep 2; 800-807
11. Allsopp R C, Vaziri H, Patterson C, et al (1992). Proc Nat Acad Sci USA 89: 10114-10118
12. Harley C B, Futcher A B, Greider C W (1990). Nature 345: 458-460
13. Oexle K, Zwirner A (1997). Human Mol Genet 6: 905-908
14. Shiels P G, Kind A J, Campbell K H, et al (1999b). Nature 399: 316-317
15. Shiels P G, Kind A J, Campbell K H, et al (1999c). Cloning 1: 1-7
16. Miyashita N, Shiga K, Yonai M, et al (2002). Biol Reprod, 66:1649-1655
17. Lanza R P, Cibelli J B, Blackwell C, et al (2000). Science 288:665-669
18. Betts D, Bordignon V, Hill J, et al (2001). Proc Nat Acad Sci USA 98 (3): 1077-1082
19. Kubota C, Yamakuchi H, Todoroki J, et al (2000). Proc Natl Acad Sci USA. 97:990-995.
20. Tian X C, Xu J, Yang X (2000). Nat Genet 26:272-273
21. Paul L C (1995). Kid Int; 56:783-793
22. Linskens M H, Feng J, Andrews W H, et al (1995). Nucleic Acids Res 23:3244-3251
23. Coles J G, Romaschin A D, Wilson G J, et al (1992). Transplantation. 54:175-178
24. Oehlschlager S, Albrecht S, Hakenberg O W, et al (2002). Luminescence; 17:130-132.
25. Daemen M A, de Vries B and Buurman W A (2002). Transplantation 73;1693-1700
26. Land W, Schneeberger H, Schleibner S, et al (1994). Transplantation 57:211-217.
27. Pollack R, Andrisevic J H, Maddux M S, et al (1993). Transplantation 55:57-60
28. Soares M P, Lin Y, Anrather J, et al (1998). Nat Med 4:1073-1077
29. Sato K, Balla J, Otterbien L, et al (2001). J Immunol 166:4185-4194
30. Joosten S A, van Ham V, Nolan C E, et al (2002). Am J Path. submitted
31. Kirk C, Nolan C E, Jardine A G, et al (2002). Am J Transpl. submitted
32. Chkhotua A, Shohat M, Tobar A, et al (2002). Transplant Immunol 9:165-171

33 Cui W, Aslam S, Fletcher J, et al (2002). J Biol Chem. 277:38531-9.

TABLE 1
Senescence associated genes
The table lists a series of genes whose products are
potential markers for senescence, through having roles in
telomere maintenance, cellular damage sensing and cellular
damage signalling and repair.

| Gene | Function |
|---|---|
| Sirtuins 1-8 | SIRT 1 - negative regulator of p53 |
| | Mutants accelerated senescence in lower |
| | organisms |
| XRCC5 | DNA repair |
| G22P1 | DNA repair |
| hPOT 1 | Telomere capping. Damage signalling |
| P21 | Cell cycle control |
| P16 | Cell cycle control |
| Collagenase | Cell surface remodelling |
| TANK 1, 2 | Telomere binding proteins |
| TRF 1, 2 | Telomere binding proteins |
| WRN | DNA repair |

Appendix 2
Telomeres and Implications for Transplantation.

The primary observations indicating imprecise genomic reprogramming in sheep derived by nuclear transfer (NT) (Shiels et al 1999a,b) have been widely contradicted by observations in cattle and mice. Extrapolation of these data, however, to chronological ageing, cellular senescence and ultimately physiological senescence, is not straightforward.

One complicating factor is that a large percentage of mammals derived via NT die during gestation, or perinatally, exhibiting imprinting errors associated with epigenetic effects and abnormal gene expression (Humpherys et al 2002). This has long term implications for cloning in relation to health, supporting the description of surviving clones as the ‘survival of the least damaged’. This has obvious implications for maverick attempts at human reproductive cloning. Pertinent to these data are reports indicating that mice derived by NT from Sertoli cells show shortened life-spans (Ogonuki et al 2002), while mice derived from Cumulus cells exhibited large body size and obesity (Tamashiro et al 2002). Significantly, this genetic dysregulation has been attributed to imprecise nuclear programming and not to in vitro culture (Miyashita et al 2002). Therapeutic cloning to provide cellular transplants, however, should be relatively less affected, though the degree to which this is so may be dependent on species and cell type.

Recent data on telomeres in cattle derived by NT, indicate that the cell type of the donor nucleus may indeed be a critical factor (Miayshita et al 2002).

Telomeres in Animals Derived by NT

Initial reports of shortened mean telomere restriction fragment (mTRF) lengths in two animals 6LL3 (Dolly), derived from ovine mammary epithelial cells, and 6LL6, derived from sheep embryonic cells, suggested that full genetic reprogramming of the nucleus was not achieved in all instances. Notably, a third sheep, 6LL7 derived from a fibroblast nucleus had normal mTRF lengths (Shiels et al 1999a,b). This situation remained unique until the recent observations by Miyashita et al (2002) demonstrating shortened mTRFs in cattle derived by NT, in comparison to age matched controls.

Previous observations on cattle derived by nuclear transfer have indicated that resetting of telomere based molecular clocks is attainable, though it is not always precise. Calves derived form fetal fibroblasts by NT showed either longer telomeres than age matched controls or equivalent lengths (Lanza et al 2000; Tian et al 2000, Kubota, et al 2000; Betts et al 2001). Normal telomere lengths have also been reported for mice (Wakayama et al 2000). The reasons for these differences are unclear and do not readily appear to be related to time spent by donor cells in culture, as near senescent cells have been used to successfully derive cloned cattle (Lanza et al 2000).

Recent observations have demonstrated significant variation in telomere lengths among cattle derived from different cell types (Miyashita et al 2002). What is notable about this work, is that animals derived from epithelial cells exhibit shorter mTRF lengths than age matched controls, while conversely, fibroblast donors resulted in animals with extended telomeres. These data, though limited, are intriguing. They are consistent with the original observation on Dolly, again derived from an epithelial cell source, having shortened mTRF lengths in comparison to age matched controls. It appears that Dolly is no longer an exception in this respect. Notably, an ovine fibroblast clone 6LL7 had normal mTRF lengths, consistent with the observations of Miyashita et al.

A number of possibilities present themselves to explain these observations. One original hypothesis to explain the discrepancies between the ovine, bovine and murine data, was simply species differences. This now appears to be unlikely. More likely, is that the process of NT leads to telomere extension and that donor cell specific differences influence the extent of this.

How might NT Influence Telomere Extension?

Developmental reprogramming, inherent in NT, should result in telomerase reactivation and repair of any somatic telomere erosion in surviving clones, unless the TERT coding sequence is adversely mutated while the donor cell is in culture, or subsequent telomerase activity is limited post NT. Insufficient telomerase activity has been demonstrated to allow primary ovine fibroblast cultures to bypass senescence. Critically, this situation is compounded by the fact that telomere shortening and consequent genomic instability are still extant in these cells (Cui et al 2002). This has significant implications for cell based transplant therapies as will be discussed below.

Why the apparent cell type differences in telomere length? One point of variance is the enucleation of the recipient oocyte. Differential removal of key cytoplasmic components might adversely affect re-establishment of telomere nucleoprotein complexes in resultant clones. Insufficient protein, or maternally derived message in the egg cytoplasm, might result in incorrect or less stable telomere complexes and thus facilitate an increased rate of attrition in the developing clone, which may impact upon the physiology of the animal.

This situation could be exacerbated by the distribution of telomere binding proteins in the donor nucleus and result in the observed cell type differences in mTRF lengths. There is precedent for such a hypothesis. TRF1, a key telomere binding protein and functional telomerase antagonist is differentially distributed according to cell type (Steensel and de Lange, 1997;Broccoli et al 1997). Other examples of differentially expressed telomere binding proteins include TNKS 1,TNKS 2,TERF1 and TERF2 (details of individual expression patterns can be found at http://bioinformatics.weizmann.ac.il/cards). It is feasible that cell cycle difference in the expression of such genes might also influence the availability of telomere binding proteins.

Another consideration is the nature and extent of the telomere repair mechanism. It remains to be determined if telomerase is solely responsible for resetting telomere lengths in animals derived by NT, or if an alternative telomere lengthening (ALT) mechanism is also contributory. Normally the two mechanisms are mutually exclusive and it is telomerase that fulfils the repair (Henson et al 2002). The telomere extension observed in fibroblast clones, including those derived from senescent cell nuclei, may again be due to enhanced access of telomerase to the chromosome ends, as a consequence of insufficient availability of key protein components in the telomere nucleoprotein complex. The contribution of any alternative lengthening of telomeres (ALT) possibly activated by imprecise reprogramming is yet to be formally assessed by experiment and is open to speculation. A recombinogenic mechanism, acting in concert with telomerase, might also be expected to produce telomere extension. This may also lead to increased chromosomal instability in such animals.

A more pertinent consideration may be the extent and timing of telomerase activity during development. It is possible that this could be contributory to the observed species differences between some NT derived animals. Ovine clone 6LL6, for example, derived from sheep embryonic cells, showed telomere shortening. Conversely, bovine embryonic cell derived clones exhibited telomere extension (Shiels et al 1999a,b; Miyashita et al 2002). NT has a previously been reported to up-regulate telomerase activity specifically in bovine embryos, which would be in keeping with reported telomere extension in bovine embryonic cell derived clones. The situation with respect to developmental timing and extent of telomerase activity in sheep remains to be determined.

Physiological Impact of Telomere Dynamics

Assessing the physiological impact of the observed telomere dynamics in surviving clones is not straightforward, given that the situation may be compounded cryptic reprogramming errors. A further consideration is species. Murine telomeres, for example, function within a telomerase positive soma and though functionally analogous to other mammalian telomeres, the precise composition of their nucleo-protein telomere complex may not be identical as a consequence. The fact that murine telomeres are substantially longer than in man is suggestive that the physiological impact of telomere erosion might be less severe, as critical telomere lengths are not reached within the animal's lifetime. Similarly, the relative telomere shortening observed in Dolly and the animals derived from bovine epithelial cells, may similarly have no real physical impact in the animals life time as critical telomere lengths are unlikely to be reached. This is supported by a comparison of critical telomere lengths in bovine cells determined in vitro and in surviving clones derived from the same cell line (Miyashita et al 2002).

This situation may not hold when transposed to a clinical transplant setting. Nuclear transfer offers great promise in facilitating the provision of cells and organs for both allo- and xeno-transplantation. Transplantation, however, induces stresses that may prematurely senesce the graft, both at the cellular and physiological levels.

This has relevance to solid organ transplantation, including xenografts from pigs derived by nuclear transfer. Transplantation can induce both apoptosis and accelerated senescence in the graft as a result of oxidative damage caused by ischaemia and reperfusion. This arises through the switch from aerobic to anaerobic metabolism in the graft upon ischaemia and back aerobic metabolism upon reperfusion. Consequently, dependent upon the number and location of cells, either lost through apoptosis or prematurely senesced, the physiological capacity of the organ will be affected. When the organ is subject to further stress (e.g. immune injury) this may be sufficient to predispose to rejection pathologies. Precedent for such a situation can be found in allo-transplantation. Premature senescence of human kidney allografts has previously been proposed to predispose to chronic rejection (Halloran and Melk 2001; Shiels et al 2002), while in a rat model of chronic kidney rejection, massive telomere erosion and premature senescence has been observed associated specifically with chronic rejection (Joosten et al 2002). Significantly, in this latter example, senescence associated changes appear to be found principally in tubular epithelium. This may reflect that epithelial cells in the kidney are under increased stress relative to surrounding cell types, or that they are less well able to withstand damage. It seems intuitive that cells with a high turnover rate, such as the epithelial cells, might have telomere nucleo-protein complexes that dissociate more readily than other types, as a damage response mechanism. To put this in context, tubular epithelium is under significant fluidic stress, which might exacerbate the relative rate of senescence in the component cells and hence physiological senescence in the organ as a whole. This is in keeping with observations on endothelial cells under elevated haemodynamic stress. These cells show accelerated telomere erosion at points of vessel bifurcation and at atherosclerotic lesions (Minamino et al 2002). It will be interesting to determine if bovine or ovine endothelial cell clones also exhibit telomere shortening.

Recently, massive and specific telomere erosion has been reported as an early marker of apoptosis induced by DNA damage (Ramirez et al 2002) in keeping with the above observations in rat kidney transplants. This loss of telomeric DNA occurs early in apoptosis and is coincident with collapse of the mitochondrial membrane potential and production of reactive oxygen species.

Accelerated senescence also impacts upon cellular transplants. Any cell population differentiated and expanded in vitro, unless the cells are telomerase positive, will also be prone to accelerated senescence. The effects of in vitro oxidant stress on the rate of telomere erosion have been well documented (Shiels 1999c; Gorbunova et al 2002). As with solid organ transplants, such telomere erosion may have a physiological consequence post transplant. This would have implications for stem cell therapies and is suggestive of in vivo differentiation of stem cells being preferable to in vitro differentiation and expansion. The risk of neoplasia occurring as a consequence of such transplants has still to be fully determined in vivo.

Conclusion: Epithelial Cell Clones

The arrival of cattle derived by nuclear transfer from epithelial cells and exhibiting shortened telomeres, means Dolly is neither an exception nor a ‘fluke’. Any physiological consequences of having shortened telomeres in all these animals is unlikely to develop in the animals life time, unless the situation is compounded by cryptic reprogramming errors as a result of NT. These observations do have implications for animals derived by NT for xenotransplantation or for cellular therapies. In such instances, transplantation may lead to accelerated cellular and physiological senescence with consequent predisposition to disease post transplant.

References

Betts D, Bordignon V, Hill J, et al. (2001) Reprogramming of telomerase activity and rebuilding of telomere length in cloned cattle. Proc Natl Acad Sci U S A.;98:1077-1082
Broccoli D, Chong L, Oelmann S, et al (1997) Comparison of the human and mouse genes encoding the telomeric protein, TRF1: chromosomal localization, expression and conserved protein domains. Hum Mol Genet. (1):69-76.
Cui W, Aslam S, Fletcher J et al. (2002) Stabilization of telomere length and karyotypic stability are directly correlated with the level of hTERT gene expression in primary fibroblasts. J Biol Chem. 277:38531-9.
Gorbunova V, Seluanov A, Pereira-Smith O M. (2002) Expression of human telomerase (hTERT) does not prevent stress-induced senescence in normal human fibroblasts but protects the cells from stress-induced apoptosis and necrosis. J Biol Chem. 277:38540-9.
Halloran P F, Melk A, Barth C. (1999) Rethinking chronic allograft nephropathy: the concept of accelerated senescence. J Am Soc Nephrol. 10:167-181.
Henson J D, Neumann A A, Yeager T R, et al (2002) Alternative lengthening of telomeres in mammalian cells. Oncogene 21:598-610.
Humpherys D, Eggan K, Akutsu H et al(2002) Abnormal gene expression in cloned mice derived from embryonic stem cell and cumulus cell nuclei. Proc Natl Acad Sci U S A. 99:12889-12894.
Joosten S A, van Ham V, Nolan C E et al (2002) Telomere shortening and cellular senescence in a model of chronic renal allograft rejection. Am J Path submitted
Kubota C, Yamakuchi H, Todoroki J et al (2000) Six cloned calves produced from adult fibroblast cells after long-term culture. Proc Natl Acad Sci U S A.;97:990-995
Lanza R P, Cibelli J B, Blackwell C, et al (2000). Extension of cell life-span and telomere length in animals cloned from senescent somatic cells. Science.;288:665-669.
Minamino T, Komuro I. 2002 Role of telomere in endothelial dysfunction in atherosclerosis. Curr Opin Lipidol. 13:537-543.
Miyashita N, Shiga K, Yonai M et al (2002). Remarkable differences in telomere lengths among cloned cattle derived from different cell types. Biol Reprod. 66:1649-55.
Ogonuki N, Inoue K, Yamamoto Y, et al (2002). Early death of mice cloned from somatic cells. Nat Genet. 30:253-4.
Ramirez R, Carracedo J, Jimenez R, et al (2002) Massive telomere loss is an early event of DNA damage-induced apoptosis. J Biol Chem. 2002 Oct. 29 E Pub ahead of print
Shiels P G, Kind A J, Campbell K H, et AL (1999a) Analysis of telomere lengths in cloned sheep. Nature 399: 316-317.
Shiels P G, Kind A J, Campbell K H, et al(1999b) Analysis of telomere length in Dolly, a sheep derived by nuclear transfer. Cloning 1(2): 1-7.
Shiels P G (1999c) Somatic cell nuclear transfer as a tool for investigating ageing processes in Mammals Gene Ther. Mol Biol 4:11-22.
Shiels P G, Nolan C E, Sklavounou E (2002) Cellular senescence, organ ageing and renal pathogenesis. Am J Transplantation (in press)
Tamashiro K L, Wakayama T, Akutsu H, et al (2002) Cloned mice have an obese phenotype not transmitted to their offspring. Nat Med. 8:262-267.
Tian X C, Xu J, Yang X. (2000) Normal telomere lengths found in cloned cattle. Nat Genet. 26:272-273.
van Steensel B, de Lange T. (1997) Control of telomere length by the human telomeric protein TRF1. Nature.; 385:740-743.
Wakayama T, Shinkai Y, Tamashiro Klet al (2000) Cloning of mice to six generations. Nature. 407:318-319.
   Appendix 3
   Cellular Senescence, Organ Ageing and Transplantation

The demand for donor cell and organ transplants continues to outstrip supply, despite the use of marginal donors and the continuing development of promising alternatives, such as xenotransplants and stem cell therapy.

Important lessons for transplant success, however, can be found through a better understanding of the molecular processes that may predispose to disease and disease state progression. It is in this context that the understanding of mechanisms of senescence may be particularly beneficial. This is especially relevant to late (chronic) transplant rejection and the use of older and marginal donors. It has been proposed that oxidative damage at the time of transplantation sets in motion a series of events that results in accelerated senescence within the graft (1-3) leaving it less able to cope with further stress. Consequently, the graft is more vulnerable to immune and non-immune mediated injury and is predisposed to disease. This mini review is designed to provide insight into current research on senescence and how it may relate to the success of transplanted cells and organs.

The effects of senescence must be viewed from three perspectives, ranging from the level of whole organism, to the organ level and the cellular level. These can be loosely summarised as follows;
Chronological ageing—the cumulative burden of environmental and metabolic insults over the time elapsed since birth. This phenotype is typified by loss of organ mass and integrity, along with declining organ function.
Cellular senescence—ageing at a cellular level. Typified by growth arrest and loss of replicative potential, due principally to oxidative damage to DNA and the oxidation of proteins and lipids.
Physiological senescence—a decline in physiological capacity at the level of the organ. This is distinct from cellular senescence, but overlapping extensively with it. Critically, the degree of physiological senescence will be dependent upon the numbers and location of cells lost through insult, apoptosis or affected by cellular senescence.

Ageing at the Molecular Level

Senescence is considered an evolutionary mechanism to counter neoplasia (4). At the cellular level it is typified by growth arrest, the erosion of telomeres and the expression of specific senescence associated genes (SAGs), (see table 1). The expression of these genes forms an integrated response to oxidant stress within the cell, that links energy production, energy utilisation and cell cycle control (see FIG. 5). Consequently, it necessitates functional ties between the mitochondrion, the production of ribosomes and the maintenance of telomere nucleoprotein complexes (4,5).

Telomeres

Telomeres are specialised nucleoprotein complexes found at the end of eukaryotic chromosomes. In mammals, they comprise a dynamic complex of proteins bound to stretches of a simple DNA repeat (TTAGGG)ₙ (6).This functions to cap the free DNA end, distinguishing it from a damage related DNA break and ensures the complete replication of chromosome ends. The telomeric DNA component of human chromosomes in somatic cells shortens with increasing age (7). This is a consequence of incomplete replication of chromosome ends and the loss of telomeric DNA via nuclease action (8). In germ cells and stem cells, however, this erosion is countered by the action of the enzyme telomerase. In the soma, telomere erosion continues unabated and is considered to provide a mitotic clock that eventually signals cell death. Telomere shortening has been causally implicated in human disease and by general implication, the physiological ageing process in higher animals (7,9,10).

The reason for this stems from the fact that telomeric proteins form part of a damage sensing and signalling system. This can either facilitate DNA repair or apoptosis, dependent on the level of damage. An inability to respond to, or repair damage properly, can result in accelerated ageing. Progeric conditions, such as Werners' syndrome, Blooms' syndrome and Hutchinson-Guilfords' disease all result from the dysfunction of an individual telomeric protein. In mice, knock out of the telomerase coding sequence resulted in progressive loss of telomeric DNA and progeria (11). Reintroduction of telomerase reversed both these effects (12).

Ageing at the Level of the Organ

Moving from observations on cellular senescence to the level of the organ is not straightforward, but is critical to understanding how senescence may impact upon transplant success. The situation is complicated by the need to factor in the number of senescent cells within the organ and critically for organ function, their location. This is further compounded by a similar need to address the number and location of cells lost either through insult or as a consequence of mass loss with chronological ageing (FIG. 5).

The development of nuclear transplantation (NT, cloning) has allowed the integration of in vitro observations on cellular senescence with those on the physiology of living organisms. Recently, it has provided significant insight into how senescence of a given cell type may be critical in predisposing to disease and how this might be reflected in the physiology of animals derived by NT. These data have substantial implications for transplantation.

Animals derived by NT from epithelial cell donor nuclei all appear to have shortened telomeres (13-15), in contrast to those derived from fibroblasts or muscle cells (15-19). This indicates that different cell types may have differing capacities to withstand and repair the effects of oxidative insult. In turn, this would influence the degree of physiological senescence in the organs of the cloned animal.

If critically short telomeres were reached in the animals' lifetime then the effects of such senescence might manifest in early onset of diseases of ageing. Observations in cloned sheep and cattle indicate that replicative senescence alone is insufficient to precipitate such a state of affairs, as the rate of telomere erosion in vivo is too slow and the animals appear to have sufficient telomeric DNA to prevent a crisis being reached in their lifetime (13-19). These animals are, for all intents and purposes, physiologically normal, but this situation may not hold true for xenotransplanted cells or organs derived from such animals, as will be discussed below.

Senescence and Chronic Allograft Nephropathy

Previous observations on the ageing kidney suggest that it shares many features in common with allografts showing late dysfunction (1,2). Late graft dysfunction (e.g. chronic allograft nephropathy, CAN) is the main cause of graft loss in the first post-transplant decade. Its pathogenesis remains undetermined, though it is thought to comprise both immune and non-immune factors. It is characterised by interstitial fibrosis, fibrotic intimal thickening of the small arteries and tubular atrophy. These features also manifest with increasing chronological age in the human kidney. Several observations support a hypothesis whereby dysfunctioning allografts exhibit features of senescence, both at the molecular and cellular level (1,20). These include;
The association of chronic allograft nephropathy (CAN) with increased donor age
Poorer transplant outcome with marginal donors
Ischaemic damage prior to organ retrieval
Similarities in the histological features of CAN and aged kidneys, including the expression of senescent-associated extra cellular matrix proteins (21).
Increased expression of the known senescence markers senescence associated beta galactosidase, p16 and p21 in a rat model of transplant dysfunction, in conjunction with accelerated telomere erosion (P G Shiels and L C Paul pers. obs.).

The observation of poorer transplant outcome associated with organs from older or marginal donors is consistent with the involvement of senescence.

Cells in senescence do not respond to external growth stimuli, though they remain metabolically active. Consequently, they will contribute to an impaired tissue integrity and persistent inflammation through senescence associated up-regulation of pro-inflammatory cytokine gene expression (22).

A decrease in the number of functioning nephrons and the ability to withstand stress, compounded by a loss of mass with chronological age, are intrinsic features of the renal senescence phenotype (1,20). Such organs may function adequately, but will be more susceptible to the effects of transplant related stress simply because they will already exhibit substantial physiological senescence and have a correspondingly reduced capacity to withstand stress and repair damage.

The outcome of a transplant to an older recipient will also be negatively affected by a decline in stress responses with increasing chronological age.

Transplant Related Stress

Stress at the time of transplantation centres on ischaemia and reperfusion (I/R) injury. Its consequences are profound and its effects potentially underestimated. I/R related oxidant stress occurs on two levels. Firstly, the switch from aerobic to anaerobic metabolism coincident with ischaemia and secondly, the switch back to aerobiosis upon reperfusion.

The generation of free radicals through I/R injury has been demonstrated previously to induce apoptosis and inflammation. These agents are readily detectable post-transplant in the serum of transplant recipients (23). Free radical damage to DNA, lipids and proteins will result as a direct consequence of their generation, which will then impact upon graft physiology. Crucially, dependent upon the level of cellular damage, either cellular senescence will ensue, if the damage to the cell is sub lethal, or apoptosis, if the damage is too great. It is reasonable to suggest that the number and location of cells lost, or damaged will be critical to subsequent organ function. Consequently, upon further insult (e.g. immune injury) the graft will be susceptible to further injury and thus disease, such as CAN.

Correspondingly, anti-oxidant therapy has been used to demonstrate a reduction in I/R injury and improved graft outcome (25). The use of superoxide dismutase (SOD) has been reported to provide a significantly improved 1 and 4 year graft survival rate and reduction in first acute rejection episodes after cadaveric renal transplantation. These data are still contentious. SOD activity inherently generates hydrogen peroxide, a notorious oxidant. Furthermore, differences in the mode of SOD administration, dosage and intracellular targeting may be responsible for yielding equivocal results. Alternatives, such as catalase or Heme oxygenase-1 (26), may be a more suitable anti-oxidant to use in this setting.

Experimental Observations

A number of lines of evidence indicate that I/R injury will result in apoptosis. Massive telomere destabilisation and altered expression of SAGs in grafts have been observed with as little as 45 minutes ischaemic time in rat model of chronic renal allograft rejection (P G Shiels and L C Paul pers obs), consistent with apoptosis and hence accelerated physiological senescence. Specific telomere erosion is an early event in apoptosis and precedes activation of the caspase cascade in the mitochondrion (5). Significantly, the classical molecular markers of cellular senescence p21 and p16 show elevated expression following reperfusion in these grafts. These data are paralleled by changes in the expression of telomere binding proteins linked to signalling and repair of DNA damage. However, senescence associated beta galactosidase expression (SA beta Gal), a classical cytological marker of senescence associated oxidant stress, is limited solely to the tubular epithelium of rejecting grafts. This has major implications for the physiological impact of transplant related stress and disease predisposition. These observations are consistent with data from a rat model of I/R injury (27). Recovery of function after I/R injury in this model correlated inversely with the extent of cellular senescence, as determined by amount of SA beta Gal staining.

The apparent enhanced susceptibility of tubular epithelial cells to stress is in keeping with two diverse observations. Firstly, animals cloned from epithelial cell show advanced telomere erosion and do not appear to fully repair senescence associated oxidative damage (13-15). Secondly, the Uric acid transporter (URAT1) is found solely on tubular epithelium in the kidney (28). Uric acid is the primary anti-oxidant in the human blood. Mutations in URAT1 are responsible for idiopathic renal hypouricaemia, a condition typified by exercise induced acute renal failure and reactive oxygen species induced tubule cell death, which is consistent with the above hypothesis. Epithelial cell senescence may therefore have a disproportionate effect upon kidney function.

Tackling Transplant Senescence

Accelerated graft senescence poses problems for both allotransplants and xenotransplants; I/R injury will be common to both. The problem for xenotransplants may even be compounded by nuclear transplantation. In this instance, a graft from an animal with shortened telomere lengths might prove to be compromised once subject to I/R injury (3). In an allotransplant setting this would imply that grafts from older donors would function less well post-transplant, as might those from cadavers. Again, this is consistent with clinical observations.

Cellular transplants have other complications. Somatic cells in culture typically exhibit accelerated senescence, inclusive of telomere erosion (4). Stem cell therapies using in vivo differentiation strategies could potentially avert this, though controlling the differentiation of stem cell transplants in vivo remains a technical challenge.

Stem cells display telomerase activity and do not show typical senescence in culture, as defined by telomere erosion and growth arrest. This description masks that fact that they will be still be subject to the effects of free radical damage in culture and that this may only manifest post transplant, potentially leading to oncogenesis. Significantly, stem cells show ex vivo p53-dependent asymmetric cell kinetics, which has senescence as its end point (29). Asymmetric growth kinetics may therefore explain some of the difficulties in expanding adult stem cell populations and may relate to decreased telomerase activity in these cells. The contribution of any mitochondrial damage in stem cells has yet to be fully assessed in such a context.

Hematopoetic stem cell transplants, however, illustrate the potential of this strategy. Apparent telomere length homeostasis is reached post transplant if the required cell expansion is not too great and the endogenous telomerase activity is sufficient. If not, senescence can ensue (30). The functional consequences of this may be a limitation in immune responsiveness.

Pertinent to this issue, is the transplantation of fully differentiated cell types bearing telomerase expression constructs to combat cellular senescence. This may be problematic. Recent data suggests that such cells may bypass senescence, but retain an unstable genome, due to insufficient telomerase activity, which could then predispose to oncogenesis (31). Control of the level of telomerase activity may thus be a critical aspect of future cellular transplants.

Mitigating I/R injury using anti-oxidant therapy pre-transplant may prove sufficiently efficacious. Targeting of the anti-oxidant to the mitochondrion may prove to be beneficial, given its relationship with the telomere-nucleoprotein complex. Anti-apoptotic agents will also prove crucial in this setting and open up a new therapeutic avenue for immunosuppressant drugs. Calcineurin inhibitors, have already been demonstrated to have some effect in this capacity (32), and the timing of their administration may be critical. Small molecule analogues could be considered for use.

A further avenue is gene therapy; possibly targeted to tubular epithelium to assist in reducing transplant related stress, or tackling areas of focal damage post-transplant.

References

1. Halloran P F, Melk A, Barth C. Rethinking chronic allograft nephropathy: the concept of accelerated senescence. J Am Soc Nephrol. 1999, 10:167-81.
2. Melk A, Halloran P F Cell senescence and its implications for nephrology. J Am Soc Nephrol. 2001, 12:385-393.
3. Shiels P G and Jardine A G Dolly, no longer the exception; telomeres and implications for transplantation. Cloning and Stem cells 2002. In press.
4 Shiels P G Somatic cell nuclear transfer as a tool for investigating ageing processes in Mammals. Gene Ther. Mol Biol 1999, 4:11-22.
5. Ramirez R, Carracedo J, Jimenez R, Canela A, Herrera E, Aljama P, Blasco M A. Massive telomere loss is an early event of DNA damage-induced apoptosis. J Biol Chem. 2002 Oct. 29 [epub ahead of print].
6. Moyzis R K, Buckingham J M, Cram L S, Dani M, Deaven L L, Jones M D, Meyne J, Ratliff R L, Wu J R. A highly conserved repetitive DNA sequence, (TTAGGG)n, present at the telomeres of human chromosomes. Proc Nat Acad Sci USA, 1988, 85:6622-6626.
7. Harley C B, Futcher, A B, Greider C W Telomeres shorten during ageing of human fibroblasts Nature 1990, 345:458-460.
8. Karlseder J, Smogorzewska A, de Lange T. Senescence induced by altered telomere state, not telomere loss. Science. 2002, 295:2446-2449.
9. Allsopp R C, Vaziri H, Patterson C, Futcher A B, Greider C W, Harley C B. Telomere length predicts replicative capacity of human fibroblasts. Proc Nat Acad Sci USA, 1992, 89:10114-10118.
10. Oexle K, Zwirner A Advanced telomere shortening in respiratory chain disorders. Human Mol. Genet. 1997, 6: 905-908.
11. Blasco M A, Lee H-W, Hande P, Samper E, Lansdorp P M, DePinho R A, Greider C W. Telomere shortening and tumor formation by mouse cells lacking telomerase RNA. Cell 1997, 91:25-34.
12. Samper E, Flores J M and Blasco M A Mammalian Ku86 protein prevents telomeric fusions independently of the length of TTAGGG repeats and the G-strand overhang. EMBO rep 2001, 2:800-807.
13. Shiels P G, Kind A J, Campbell K H, Waddington D, Wilmut I, Colman A, Schnieke A E Analysis of telomere lengths in cloned sheep. Nature 1999, 399:316-317
14. Shiels P G, Kind A J, Campbell K H, Waddington D, Wilmut I, Colman A, Schnieke A E Analysis of telomere length in Dolly, a sheep derived by nuclear transfer. Cloning 1999, 1:1-7.
15. Miyashita N, Shiga K, Yonai M, Kaneyama K, Kobayashi S, Kojima T, Goto Y, Kishi M, Aso H, Suzuki T, Sakaguchi M, Nagai T Remarkable differences in telomere lengths among cloned cattle derived from different cell types. Biol Reprod. 2002, 66:1649-1655.
16 Lanza R P, Cibelli J B, Faber D, Sweeney R W, Henderson B, Nevala W, West M D, Wettstein P J. Cloned cattle can be healthy and normal. Science. 2001, 294:1893-1894.
17. Tian X C, Xu J, Yang X. Normal telomere lengths found in cloned cattle. Nat Genet. 2000, 26:272-273.
18. Betts D, Bordignon V, Hill J, Winger Q, Westhusin M, Smith L, King W. Reprogramming of telomerase activity and rebuilding of telomere length in cloned cattle. Proc Natl Acad Sci U S A. 2001, 98:1077-1082
19. Kubota C, Yamakuchi H, Todoroki J, Mizoshita K, Tabara N, Barber M, Yang X. Six cloned calves produced from adult fibroblast cells after long-term culture. Proc Natl Acad Sci U S A. 2000, 97:990-995
20. Paul L C Chronic renal transplant loss. Kid Int 1995, 56:783-793
21. Linskens M H, Feng J, Andrews W H, Enlow B E, Saati S M, Tonkin L A, Funk W D, Villeponteau B. Cataloging altered gene expression in young and senescent cells using enhanced differential display. Nucleic Acids Res. 1995, 23:3244-3251.
22. Chung H Y, Kim H J, Kim J W, Yu B P. The inflammation hypothesis of aging: molecular modulation by calorie restriction. Ann N Y Acad Sci. 2001, 928:327-335.
23 Oehlschlager S, Albrecht S, Hakenberg O W, Manseck A, Froehner M, Zimmermann T, Wirth M P. Measurement of free radicals and NO by chemiluminescence to identify the reperfusion injury in renal transplantation. Luminescence 2002, 17:130-132.
24 Lieberthal W, Triaca V, Koh J S, Pagano P J, Levine J S. Role of superoxide in apoptosis induced by growth factor withdrawal. Am J Physiol 1998, F315-327.
25 Land W, Schneeberger H, Schleibner S, Illner W D, Abendroth D, Rutili G, Arfors K E, Messmer K The beneficial effect of human recombinant superoxide dismutase on acute and chronic rejection events in recipients of cadaveric renal transplants. Transplantation. 1994, 57:211-217.
26 Soares M P, Lin Y, Anrather J, Csizmadia E, Takigami K, Sato K, Grey S T, Colvin R B, Choi A M, Poss K D, Bach F H. Expression of heme oxygenase-1 can determine cardiac xenograft survival. Nat Med 1998, 4: 1073-1077
27. Chkhotua A, Shohat M, Tobar A, Magal N, Kaganovski E, Shapira Z, Yussim A. Replicative senescence in organ transplantation-mechanisms and significance. Transplant Immunol 2002, 9: 165-171
28 Enomoto A, Kimura H, Chairoungdua A, Shigeta Y, Jutabha P, Cha S H, Hosoyamada M, Takeda M, Sekine T, Igarashi T, Matsuo H, Kikuchi Y, Oda T, Ichida K, Hosoya T, Shimokata K, Niwa T, Kanai Y, Endou H. Molecular identification of a renal urate anion exchanger that regulates blood urate levels. Nature.2002, 417:447-452
29 Rambhatla L, Bohn S A, Stadler P B, Boyd J T, Coss R A, Sherley J L. Cellular Senescence: Ex Vivo p53-Dependent Asymmetric Cell Kinetics. J Biomed Biotechnol. 2001, 1:28-37.
30. Roelofs H, De Pauw E S, Zwinderman A H, Opdam S M, Willemze R, Tanke H J, Fibbe W E. Homeostasis of telomere length rather than telomere shortening after allogeneic peripheral blood stem cell transplantation. Blood. 2003, 101:358-62.
31 Cui W, Aslam S, Fletcher J Wylie D, Clinton M, Clark A J. Stabilization of telomere length and karyotypic stability are directly correlated with the level of hTERT gene expression in primary fibroblasts. J Biol Chem. 2002, 277:38531-38539.

32 C Yang C W, Ahn H J, Han H J, Kim W Y, Li C, Shin M J, Kim S K, Park J H, Kim Y S, Moon I S, Bang B K. Pharmacological preconditioning with low-dose cyclosporine or FK506 reduces subsequent ischemia/reperfusion injury in rat kidney. Transplantation. 2001, 72:1753-1759.

TABLE 1
Genes showing critical expression changes with cellular
senescence.

| Telomere | | | | |
|---|---|---|---|---|
| binding | Cell | | | |
| proteins/DNA | cycle | Cellular | Transcription | |
| repair | control | Repair | factors | Others |
| SIRT1-8 | P16 | Collagenase | NF-kappaB, | Bcl-2 |
| HPOT1 | P21 | Caveolin | AP-1, | ERK |
| XRCC5 | | APO | HIF-1 | JNK |
| G22P1 | | J/Clusterin | | P38MAPK |
| TANK 1, 2 | | ARFUS1 | | NF- |
| TRF 1, 2 | | APA-1 | | kappaB, |
| WRN | | | | IL-beta, |
| | | | | IL-6, |
| | | | | TNFalpha |
| | | | | Cox-2 |
| | | | | i NOS |
| | | | | XOD |

Appendix 4 Joosten et al Am J Path 2003—in press
Telomere Shortening and Cellular Senescence in a Model of Chronic Renal Allograft Rejection.

Simone A. Joosten*, Vanessa van Ham, Claire E. Nolan¹, Maria C. Borrias, Alan G. Jardine¹, Paul)G. Shiels¹, Cees van Kooten and Leendert C. Paul.

Department of Nephrology, Leiden University Medical Center, Leiden, The Netherlands and ¹Department of Surgery, University of Glasgow, Glasgow, United Kingdom

Address correspondence to:
Simone A. Joosten, MSc
Department of Nephrology, C3P
Leiden University Medical Center
Albinusdreef 2
2333 ZA Leiden, The Netherlands
Phone: +31 71 5263964
Fax: +31 71 5248118
S.A.Joosten@LUMC.nl

Number of pages: 24 text pages and 8 pages with figures

Abbreviated title: Cellular senescence in renal transplantation

This work was supported by a grant from the Dutch Kidney Foundation (grant C98.1783), CN is in receipt of a British Transplantation training fellowship, PS and AJ are supported by Darlinda's Charitable trust.

Abstract

Cellular senescence has been suggested to play a role in the deterioration of renal graft function and has been linked to telomere shortening. We have investigated markers of cellular senescence in the F344 to LEW rat model of chronic renal transplant rejection. Syngeneic and LEW to F344 transplants were used as controls. Substantial telomere shortening was observed in all transplants, including allogeneic and syngeneic grafts from day 7 post transplant onwards. Ischemia of native F344 kidneys was already sufficient to induce telomere shortening. It is known that shortened telomeres can activate cell cycle regulators, such as p21 and p16. Accordingly, all cases showed a transient p21 increase, with a maximum at day 7 and a sustained expression of p16. Importantly, senescence associated β-galactosidase staining, a cytological marker for senescence, was only observed in tubular epithelial cells of chronically rejecting F344 allografts from day 30 post transplantation onwards. Long-term surviving LEW allografts or syngeneic F344 grafts were negative for senescence associated β-galactosidase. In conclusion, ischemia during transplantation results in telomere shortening and subsequent activation of p21 and p16, whereas senescence associated β-galactosidase staining is only present in chronically rejecting kidney grafts.

Introduction

Chronic allograft rejection (CR) is the most important cause of renal transplant loss. Various immunological and non-immunological factors have been implicated in its pathogenesis¹ The pathological lesions observed in chronic rejection and allograft nephropathy overlap with the changes observed in ageing kidneys² and it has been suggested that senescence of renal cells might contribute to the deterioration of graft function³. Somatic cells in vivo have a limit in their replicative capacity termed the Hayflick limit⁴. This limit has been ascribed to the loss of telomeric sequences at the end of chromosomes. Loss of telomeric repeats (TTAGGG) during sequential replications eventually compromises telomere function, leading to chromosomal instability and loss of genetic information. After cells have reached their maximal replicative potential they stop proliferating and may become senescent. Senescent cells are irreversibly arrested in the G1 phase of the cell cycle. Cells in senescence do not respond to various external stimuli, but remain metabolically active and contribute to an impaired tissue integrity and persistent inflammation⁵. In vivo cellular senescence has been proposed to act as a mechanism to prevent neoplastic transformation of cells. Furthermore, it is thought to act as a homeostatic mechanism to prolong the cellular lifespan^{6;7}.

Senescent cells display several characteristics, including shortened telomeres, increased expression of specific tumor suppressor genes and an increased activity of senescence associated β-galactosidase (SA β-gal)^{8;9}. In addition, alterations in cell shape, altered matrix metalloproteinase and cytoskeletal collagen expression have been described^{10;11}. Telomere erosion associated with senescence of somatic cells in culture has been extrapolated to the features of chronological ageing, including a decrease in physiological capacity, loss of mass and decreased resistance to stress. Relatively short telomeres in old kidneys^{2;12} may predispose to impaired graft outcome post transplant. One important observation, in this respect, is that kidneys from older donors show worse graft survival¹³.

The molecular mechanism by which telomere erosion limits proliferative potential has not been elucidated and a number of equivocal hypotheses have been proposed. One hypothesis is that critically shortened telomeres are unable to recruit sufficient telomeric proteins to form a functional nucleoprotein cap, which would expose a free broken DNA end as a consequence. Alternatively, a shortened telomere repeat stretch or in increase in the availability of free telomeric proteins arising trough loss of substrate sites resulting in the necessary signals for senescence¹⁴.

As cells become senescent there are accompanying changes in the expression of p21 and p16, which are involved in an arrest in the G1 phase of the cell cycle. These are induced in response to DNA damage (e.g. via p53 activation), which subsequently may activate members of the cyclin dependent kinase (CDK) inhibitor family¹⁵. Activation of p53 results in activation of inhibitors of CDK4 (INK4 family, including p16^{INK4}) and inhibitors of the cyclin E and A dependent kinases (Cip/Kip family, p21^{CiP1, Waf1, Sdi1})¹⁶. Activation of INK4 and Kip family members results in inhibition of the cell cycle in the G1 phase via the retinoblastoma protein. An increased expression of p21 is involved in the induction of senescence whereas p16 accumulates in senescent cells and is involved in maintenance of senescence¹⁶⁻¹⁸. A marker suggested to be specific for senescent cells is accumulation of lysosomal senescence associated β-galactosidase (SA β-gal) which is active at pH 6.0^{8;9}. In vitro observations indicate that SA β-gal accumulates as cells senesce, though in vivo observations supporting this are limited^{19;20}.

In the present study, we used a rat model of human chronic transplant rejection to investigate to what extent the senescence markers are present in renal allografts. Transplantation of a F344 kidney into an LEW recipient results after a brief episode of acute rejection in chronic rejection²¹. Transplantation of a LEW kidney into a F344 recipient also results in acute rejection but chronic rejection does not occur. In this model we demonstrate telomere shortening, and subsequent expression of p21 and p16 proteins in both F344 to LEW and LEW to F344 allografts. However, SA β-gal staining was only found in tubular epithelial cells of F344 allografts with chronic rejection and not in syngeneic or LEW allografts. Thus, in this-rat model of chronic rejection we found that telomere shortening is a first step for cellular senescence and occurs due to ischemia but additional processes are required for the induction of SA β-gal accumulation.

Methods

Animals

Male inbred Fisher (F344, RT1^{lv1}) and Lewis (LEW, RT1¹) rats at 9 weeks of age were purchased from Harlan, Horst, The Netherlands. Animals had free access to water and standard rat chow. Animal care and experimentation were undertaken in accordance with the National Institutes of Health Guide for the care and use of laboratory animals.

Kidney Transplantation

Kidney transplantations were performed as previously described²². Rats were 9-11 weeks of age at the time of transplantation. The left kidney of the recipient was removed and a donor kidney was transplanted in the orthotopic position. The remaining native right kidney was removed 7 days after transplantation. Postoperatively, animals received 1 mg/kg body weight of Temgesic subcutanously (buprenorphine-hydrochlorid, Schering-Plough B. V., Amstelveen, The Netherlands) for pain relief. Cold ischemia times varied between 30 and 45 minutes.

LEW rats that received a F344 kidney graft were sacrificed on day 7, 14, 30, 60 and 100 after transplantation and kidneys were perfused with PBS and snap frozen in liquid nitrogen. Similarly, F344 rats received a LEW kidney and were sacrificed on days 7, 14, 30, 60 and 100. In addition syngeneic transplants, both F344 to F344 and LEW to LEW, were performed and sacrificed on day 14 and day 60 post transplantation (post Tx). All experiments were performed in the absence of immunosuppression.

Ischemia-Reperfusion Experiments

Male F344 rats (9 weeks of age) were operated under halothane anesthesia. The renal artery was clamped using small artery clamps for 45 minutes (warm ischemia). From each rat both kidneys were clamped for 45 minutes, one kidney was removed after 45 minutes of ischemia, the second kidney was reperfused for 2 hours. Kidneys were harvested and snap frozen in liquid nitrogen.

Histology

Tissue samples were fixed in methyl Carnoy's solution, paraffin embedded and stained with periodic acid-Schiff or hematoxylin and eosin. Sections were scored blindly by a pathologist according to the Banff working classification²³.

Telomere Length Assay

To determine total telomeric DNA content the TeloREAD™ assay was used (Promega Corporation, Wisconsin, USA) as described²⁴. Briefly, total genomic DNA was isolated according to standard phenol/chloroform extraction methods. Ten nanograms of DNA were denatured and incubated with TeloDetection enzymes and a telomere specific probe, pyrophosphorolysis was performed at 55° C. for 60 minutes. Finally ENLITEN® reagent was added and light output was measured in a luminometer. Telomere lengths calculated by the TeloREAD™ assay were verified by comparison with independent control samples of known telomere lengths determined by standard Southern blot analyses of telomere restriction fragment lengths (TRF).

Protein Isolation and Western Blot Analysis.

To detect protein levels of the cell cycle regulators p21 and p16 in renal allografts Western Blot analysis was performed. Pieces of kidney cortex were homogenized using an ultra turrax (IKA labortechnik, Staufen, Germany) and lysed in lysisbuffer, containing 20 mM Tris-HCl (pH 7.4), 137 mM NaCl, 10% glycerol, 1% Triton X-100, 2 mM EDTA, 1 mM PMSF, 2 μg/ml leupeptin, 2 μg/ml antipain, 2 μg/ml chymostatin and 5 U/ml trasylol. The amount of protein was determined using the BCA protein assay (Pierce Chemical Co., Rockford, Ill.) and 70 μg of protein was applied under reducing conditions to 12% (p21) or 15% (p26) SDS-PAGE gels. After electrophoresis, proteins were blotted semi-dry to polyvinyldene fluoride (PVDF) membranes (Immobilon-P, Millipore, Bedford, USA). Membranes were blocked with PBS/0.05% Tween 20/2% casein before incubation with the primary antibody. For staining of p21 protein we used goat antibodies directed against p21 (sc-397G, Santa Cruz Biotechnology, California, USA) that were subsequently incubated with a HRP conjugated rabbit anti goat antiserum (Dako, Glostrup, Denmark). To detect p16 protein we used a mouse monoclonal antibody (sc-1661, Santa Cruz) followed by a HRP conjugated goat-anti-mouse antiserum (Dako). Finally, development of the blots was performed with Supersignal (Pierce) and exposure to Hyperfilm™ films (Amersham Pharmacia Biotech, UK).

Blots were stripped using Restore™ Western Blot Stripping buffer (Pierce) for 20 minutes at 37° C. Subsequently blots were incubated with a mouse monoclonal antibody reactive with actin (sc-8432, Santa Cruz) followed by a HRP conjugated goat-anti-mouse antiserum (Dako) to quantify the amount of proteins in all samples.

Expression was quantified using Stratagene-EagleSight software (Stratagene, Amsterdam, The Netherlands)

Immunohistochemistry

Detection of p16 protein expression was performed with immunohistochemistry using acetone fixed, 3 μm thin cryostat sections. First the endogenous peroxidase activity and non-specific protein binding were blocked using 0.6% H₂O₂/0.01% NaN₃ in PBS and PBS/1% BSA/1% normal goat serum respectively. The primary antibody, a mouse monoclonal antibody against p16 (Santa Cruz) diluted in PBS/1% BSA was applied overnight. After washing, the sections were incubated with the secondary antibody, HRP conjugated goat-anti-mouse immunoglobulins (Dako). Finally, the sections were stained with diaminobenzidine hydrochloride (DAB, Sigma) and embedded in entellan (Merck, Darmstadt, Germany).

Senescence Staining

Staining for senescence associated β-galactosidase (SA β-gal) activity was performed as previously described⁸ with some modifications. Briefly, cryostat sections (3 μm) of snap. frozen post Tx and normal kidneys were fixed with 0.2% glutaraldehyde and 2% formaldehyde. After washing with PBS, sections were incubated for 18 hours with freshly prepared stain solution at 37° C. (no CO₂). The stain solution contained 2 mg/ ml 5-bromo-4-chloro-3-indolyl β-D-galactopyranoside (X-gal, Sigma Chemical Company, St. Louis, Mo., USA) in 40 mM citric acid/sodium phosphate pH 6.0, 5 mM potassium ferrocyanide, 5 mM potassium ferricyanide, 150 mM NaCl, 30 mM MgCl₂. Subsequently, sections were washed and counterstained with eosin.

Quantification of SA β-gal staining was performed using a Zeiss microscope equipped with a full color 3 CCD camera. Images were analyzed with KS-400 image analysis software from Zeiss Kontron to quantify the amount of blue staining within the cells. Damaged tissue, vessels and glomeruli were excluded from the analysis manually. All sections analyzed were stained at the same time and all analysis parameters were kept identical.

Results

Telomere Shortening is found in both F344 and LEW Allografts from Day 7 Post Transplant.

F344 renal allografts removed from LEW recipients at various time points post Tx showed interstitial infiltrate and various glomerular lesions. Tubular atrophy and vascular lesions were present in kidneys removed at day 30 post Tx and at later time points (FIG. 6). In contrast, LEW allografts removed from F344 recipients did not show tubular atrophy or vascular abnormalities at any time point.

Telomere lengths were measured using the TeloREAD™ assay²⁴ in F344 to LEW and LEW to F344 renal allografts and compared these to control, non-transplanted LEW and F344 kidneys. Normal rat kidneys have median telomere lengths of about 45 kb (FIG. 7A). F344 to LEW and LEW to F344 allografts removed at day 60 post Tx both have shortened telomeres of about 10-20 kb (FIG. 7A). Furthermore, F344 to LEW renal allografts removed at day 7 post Tx already have shortened telomeres (FIG. 7B). The shortening of telomeres found at all later time points (including day 100) was comparable to telomere lengths found in day 7 kidneys (FIG. 7B). LEW allografts removed from F344 rats at comparable time points showed similar telomere shortening (data not shown).

Transient expression of p21 and subsequent expression of p16 in both F344 and LEW allografts.

Expression of the cell cycle regulatory proteins, like p21, has been described to be the consequence of telomere shortening. Therefore we performed Western blot analysis on lysates of F344 and LEW renal allografts and determined the p21 protein levels. In normal F344 or LEW kidneys no p21 protein was detected by Western blot analysis (FIG. 8A), whereas in both cases p21 protein level was increased by day 7 after Tx and decreased thereafter (FIG. 8B).

Expression of p16 has been suggested to increase after p21 protein levels decreased and to remain elevated during senescence^{17;18.} Normal F344 or LEW kidneys do not show p16 expression. In F344 to LEW renal allografts nuclear expression of p16 was found in tubular epithelial cells (FIG. 9A). In addition tubular basement membrane staining is found, but this is also observed in a control without the primary antibody and therefore considered non-specific (FIG. 9A). Expression of p16 in the F344 to LEW renal allografts was observed from day 7 and increased with time up to day 60 (FIG. 9B). In LEW to F344 allografts p16 was detectable from day 14 and increased with time, remaining at an elevated level up to day 60 (FIG. 9B).

SA β-gal Staining Parallels with Chronic Rejection.

F344 to LEW renal allografts and LEW to F344 allografts were stained with SA β-gal at pH 6.0. In normal F344 kidneys SA β-gal positive cells were found sporadically whereas in F344 to LEW renal allografts by day 60 after Tx increased numbers of SA β-gal positive cells were present (FIG. 10A). In F344 allografts SA β-gal positivity was mainly observed in tubular epithelial cells and never in glomerular cells. In contrast, LEW to F344 renal allografts by day 60 after Tx showed hardly any SA β-gal positive cells. Quantification of the amount of SA β-gal staining showed that LEW to F344 and normal kidneys had comparable numbers of SA β-gal positive cells. The F344 to LEW renal allografts had an increased number of SA β-gal positive cells at day 60 (FIG. 10B).

Since the F344 to LEW renal allografts showed an increased SA β-gal staining at day 60 post Tx, we investigated several other time points. F344 allografts have an increased SA β-gal staining from day 30 post Tx, which further increased until day 100. Quantification of this staining clearly shows that there is an increase in SA β-gal positive cells in time after Tx in rejecting F344 allografts (FIG. 10C).

Syngeneic F344 and LEW grafts do not show SA β-gal staining, ischemia is sufficient to induce telomere shortening.

In addition to allogeneic transplantations we performed syngeneic F344 to F344 and LEW to LEW renal transplantations and determined TRF lengths and SA β-gal staining. Syngeneic F344 to F344 transplants showed hardly any SA β-gal positive cells at day 60 after Tx (FIG. 11A). However, the TRF length of syngeneic renal grafts was as short as the TRF length of allogeneic F344 or LEW grafts at day 60 (FIG. 11B).

Since both allogeneic and syngeneic transplants already at day 7 post transplant were characterized by shortened telomeres, we investigated whether ischemia or reperfusion injury was sufficient to induce telomere shortening. Clamping of the renal artery for 45 minutes was enough to induce substantial telomere shortening which was not further increased by subsequent reperfusion (FIG. 12).

Discussion

We investigated characteristics of cellular senescence in the F344 to LEW model of chronic renal graft rejection. In both F344 and LEW allografts, as well as syngeneic grafts, telomere shortening occurred. This resulted in activation of the cell cycle regulators p21 and p16 in F344 and LEW allografts. Interestingly, only F344 allografts show SA β-gal staining, the cytological marker for cellular senescence. LEW allografts or syngeneic transplants do not show SA β-gal staining despite comparable telomere shortening. Therefore we postulate that an additional (perhaps immunological/physiological) event contributes to the development of cellular senescence. Although telomere shortening can be a trigger for senescence, it is not sufficient for the induction of SA β-gal staining in a rat kidney transplantation model (FIG. 13).

These transplantation data are remarkable, both for the degree of observed telomere loss and the rapidity of its onset. The degree of loss is too great to be accounted for by proliferation. It most likely reflects DNA fragmentation due to reactive oxygen species, as it is observed after only 45 minutes of ischemia.

Ischemia and reperfusion during transplantation result in a transient increase of reactive oxygen species in the organ, which are potent inducers of DNA breaks. The ability of reactive oxygen species to damage DNA may be related to site specific Fenton reactions. Telomeres are G-rich and therefore particularly sensitive to DNA oxidation and thus single strand breaks^{25;26}. In the F344 to LEW rat model we observed a decrease in TRF length, from approximately 45 kb to 20 kb after ischemia. This loss of approximately 25 kb is consistent with rapid telomere shortening as a consequence of oxidative damage and disruption of nucleoprotein complexes^{27;28}. This situation is exacerbated by the fact that telomere binding proteins are also involved in DNA repair. General chromosomal breaks induced by oxidative stress might attract these proteins from the telomeres, resulting in stabilization of telomeres.

Such rapid and specific telomere loss is not without precedent. Similar massive telomere loss has also been reported as an early event in DNA damage induced apoptosis in lymphocytes²⁹. Although we cannot compare lymphocytes and tubular epithelial cells directly, similar mechanisms might occur also in tubular epithelial cells. Apoptosis of tubular epithelial cells has been found after ischemia and reperfusion of the kidney³⁰. The SA β-gal staining seems to parallel with the histological and functional presence of chronic rejection in the F344 to LEW model. SA β-gal staining was observed only in F344 renal allografts transplanted in LEW recipients and not in LEW allografts transplanted in F344 rats. In addition F344 syngeneic grafts did not show SA β-gal staining, although both LEW to F344 and syngeneic grafts have shortened telomeres. Apparently acute rejection episodes that occur in both F344 to LEW and LEW to F344 transplants are not enough to induce cells to go into senescence (defined as SA β-gal positivity), even when they already have decreased TRF lengths. F344 to LEW renal transplants develop signs of CR after day 30, the time frame within which the first SA β-gal positive cells were detected. Since LEW to F344 allografts do not show increased SA β-gal positivity we believe that additional (immunological/physiological) events are required. Previously, we have demonstrated an humoral response against kidney proteins in LEW recipients of F344 grafts, that is not present in F344 recipients of LEW allografts or in recipients of syngeneic grafts²². We cannot exclude that factors involved in the induction of these immunological responses contribute to the accumulation of SA β-gal, but the presence of telomere erosion, p16 and p21 expression are consistent with its induction as a function of cellular senescence.

In the F344 to LEW renal allografts tubular atrophy, a histopathological feature of CR, was present from day 30 post TX. Furthermore, SA β-gal accumulation is mainly found in tubular epithelial cells and not in glomeruli. In patients, ischemia/reperfusion injury can result in delayed graft function. Recently it has been shown that a defect in tubular function is responsible for delayed graft function³¹. This supports the hypothesis that tubular epithelial cells are extremely sensitive to oxidative damage. In addition, in ageing human kidneys the decrease in telomere length is faster in cortex compared to medulla². In the TRF length assay cortical DNA was used, which consists predominantly of tubular cell DNA. Therefore the decreased TRF length is most likely a result of loss of tubular telomeric DNA. This implies that predominantly tubular epithelial cells are damaged by oxidative damage and that this contributes to the deterioration of graft function.

Senescence of tubular epithelial cells implies an inappropriate response to damage signals, resulting in persistent inflammation and thereby scarring. In case of senescence, injured tissue cannot be replaced by healthy epithelium and fibrosis may be a consequence³². Recently, expression of p21 has been associated with early chronic liver allograft rejection. P21 expression was predominantly found in biliary epithelial cells and was increased in patients with early CR³³. Replicative senescence of the epithelial cells was responsible for the characteristic phenotypic changes observed. In our model, p21 expression is transiently found in both F344 to LEW and LEW to F344 allografts, however this increase was not sufficient to induce SA β-gal accumulation. Tubular epithelial cells in F344 grafts with CR become SA β-gal positive, thus impaired function of tubular epithelial cells might contribute to decreased renal function.

In humans, in vivo telomere shortening with age has been demonstrated and in human renal transplantation older donor kidneys have an inferior prognosis^{12;13}. Since the transplantation procedure might induce telomere erosion it is conceivable that if telomeres are already shortened in ageing kidneys and the kidneys receive an additional stress during ischemia, the outcome will be worse. However data obtained in rodents cannot be extrapolated to humans without any precaution. Although rodents are frequently used as experimental model to study human diseases it is known that they are different in some ways. In senescence research mice and man have been shown to have a different pathogenesis³⁴, whereas in rats it is not yet clear.

Patients with CR have increased oxidative stress markers and decreased anti-oxidants suggesting that oxidative stress plays a role in the development or progression of CR^{35;36}. Ischemia/reperfusion of organ transplants results in generation of free radicals, inducing DNA breaks and telomere erosion. To prevent telomere erosion and thereby the first prerequisite for the senescent phenotype, generation of free radicals should be prevented. The usage of antioxidants in kidney preservation solutions might be helpful in preventing oxidative damage in transplant organs and influence long-term function^{37;38}. Expression of anti-oxidants can prevent lesions of CR after antibody transfer. However, the timing of anti-oxidant expression is important³⁹.

In conclusion, oxidative damage during transplantation results in damage to genomic DNA, leading to a decreased telomere length and thereby activation of the cell cycle regulators. Shortening of telomeres will not be solely sufficient for cells to become SA β-gal positive, though this will be affected by yet unknown factors.

Acknowledgements

The authors thank Dr. H. Benediktsson for scoring of the kidney histology, Dr. M. G. A. van Dixhoorn for helpful discussions and Prof. Dr. M. R. Daha for critical reading of the manuscript.

References

1. Paul L C: Chronic renal transplant loss. Kidney Int 1995, 47:1491-1499
2. Melk A, Ramassar V, Helms L M, Moore R, Rayner D, Solez K, Halloran P F: Telomere shortening in kidneys with age. J Am Soc Nephrol 2000, 11:444-453
3. Melk A, Halloran P F: Cell senescence and its implications for nephrology. J Am Soc Nephrol 2001, 12:385-393
4. Hayflick L, Moorhead P S: The serial cultivation of human diploid cell strains. Exp Cell Res 1961, 25:585-621
5. Serrano M, Blasco M A: Putting the stress on senescence. Curr Opin Cell Biol 2001, 13:748-753
6. Shiels P G, Nolan C E, Sklavounou E: Cellular senescence, organ ageing and renal pathogenesis. Am J Transplant 2002, in press
7. Shiels P G: Somatic cell nuclear transfer as a tool for investigating ageing processes in mammals. Gene Ther Mol Biol 1999, 4:11-22
8. Dimri G P, Lee X, Basile G, Acosta M, Scott G, Roskelley C, Medrano E E, Linskens M, Rubelj I, Pereira-Smith O: A biomarker that identifies senescent human cells in culture and in aging skin in vivo. Proc Natl Acad Sci U S A 1995, 92:9363-9367
9. Kurz D J, Decary S, Hong Y, Erusalimsky J D: Senescence-associated (beta)-galactosidase reflects an increase in lysosomal mass during replicative ageing of human endothelial cells. J Cell Sci 2000, 113 (Pt 20):3613-3622
10. Dai C Y, Enders G H: p16 INK4a can initiate an autonomous senescence program. Oncogene 2000, 19:1613-1622
11. Linskens M H, Feng J, Andrews W H, Enlow B E, Saati S M, Tonkin L A, Funk W D, Villeponteau B: Cataloging altered gene expression in young and senescent cells using enhanced differential display. Nucleic Acids Res 1995, 23:3244-3251
12. Yang L, Suwa T, Wright W E, Shay J W, Hornsby P J: Telomere shortening and decline in replicative potential as a function of donor age in human adrenocortical cells. Mech Ageing Dev 2001, 122:1685-1694
13. De Fijter J W, Mallat M J, Doxiadis I I, Ringers J, Rosendaal F R, Claas F H, Paul L C: Increased immunogenicity and cause of graft loss of old donor kidneys. J Am Soc Nephrol 2001, 12:1538-1546
14. Karlseder J, Smogorzewska A, De Lange T: Senescence induced by altered telomere state, not telomere loss. Science 2002, 295:2446-2449
15. Itahana K, Dimri G, Campisi J: Regulation of cellular senescence by p53. Eur J Biochem 2001, 268:2784-2791
16. Sherr C J, Roberts J M: CDK inhibitors: positive and negative regulators of G1-phase progression. Genes Dev 1999, 13:1501-1512
17. Robles S J, Adami G R: Agents that cause DNA double strand breaks lead to p16INK4a enrichment and the premature senescence of normal fibroblasts. Oncogene 1998, 16:1113-1123
18. Stein G H, Drullinger L F, Soulard A, Dulic V: Differential roles for cyclin-dependent kinase inhibitors p21 and p16 in the mechanisms of senescence and differentiation in human fibroblasts. Mol Cell Biol 1999, 19:2109-2117
19. Minamino T, Miyauchi H, Yoshida T, Ishida Y, Yoshida H, Komuro I: Endothelial cell senescence in human atherosclerosis: role of telomere in endothelial dysfunction. Circulation 2002, 105:1541-1544
20. Chkhotua A, Shohat M, Tobar A, Magal N, Kaganovski E, Shapira Z, Yussim A: Replicative senescence in organ transplantation-mechanisms and significance. Transpl Immunol 2002, 9:165-171
21. Paul L C: Experimental models of chronic renal allograft rejection. Transplant Proc 1995, 27:2126-2128
22. Joosten S A, Van Dixhoorn M G A, Borrias M C, Benediktsson H, Van Veelen P A, Van Kooten C, Paul L C: Antibody response against perlecan and collagen types IV and VI in chronic renal allograft rejection in the rat. Am J Pathol 2002, 160:1301-1310
23. Racusen L C, Solez K, Colvin R B, Bonsib S M, Castro M C, Cavallo T, Croker B P, Demetris A J, Drachenberg C B, Fogo A B, Furness P, Gaber L W, Gibson I W, Glotz D, Goldberg J C, Grande J, Halloran P F, Hansen H E, Hartley B, Hayry P J, Hill C M, Hoffman E O, Hunsicker L G, Lindblad A S, Yamaguchi Y: The Banff 97 working classification of renal allograft pathology. Kidney Int 1999, 55:713-723
24. Learish R D, Shultz J, Ho S, Bulleit R F: A small-scale telomere repeat sequence content assay using pyrophosphorolysis coupled to ATP detection submitted for publication 2002,
25. Von Zglinicki T, Saretzki G, Docke W, Lotze C: Mild hyperoxia shortens telomeres and inhibits proliferation of fibroblasts: a model for senescence? Exp Cell Res 1995, 220:186-193
26. Von Zglinicki T: Oxidative stress shortens telomeres. Trends Biochem Sci 2002, 27:339-344
27. Honda S, Hjelmeland L M, Handa J T: Oxidative stress-induced single-strand breaks in chromosomal telomeres of human retinal pigment epithelial cells in vitro. Invest Ophthalmol Vis Sci 2001, 42:2139-2144
28. Proctor C J, Kirkwood T B: Modelling telomere shortening and the role of oxidative stress. Mech Ageing Dev 2002, 123:351-363
29. Ramirez R, Carracedo J, Jimenez R, Canela A, Herrera E, Aljama P, Blasco MA: Massive telomere loss is an early event of DNA damage-induced apoptosis. J Biol Chem 2002, in press:
30. Daemen M A, De Vries B, Buurman W A: Apoptosis and inflammation in renal reperfusion injury. Transplantation 2002, 73:1693-1700
31. El Maghraby T A, Boom H, Camps J A, Blokland K A, Zwinderman A H, Paul L C, Pauwels E K, De Fijter J W: Delayed graft function is characterized by reduced functional mass measured by (99m)Technetium-mercaptoacetyltriglycine renography. Transplantation 2002, 74:203-208
32. Halloran P F, Melk A, Barth C: Rethinking chronic allograft nephropathy: the concept of accelerated senescence. J Am Soc Nephrol 1999, 10:167-181
33. Lunz J G, III, Contrucci S, Ruppert K, Murase N, Fung J J, Starzl T E, Demetris A J: Replicative senescence of biliary epithelial cells precedes bile duct loss in chronic liver allograft rejection: increased expression of p21(WAF1/Cip1) as a disease marker and the influence of immunosuppressive drugs. Am J Pathol 2001, 158:1379-1390
34. Sherr C J, DePinho R A: Cellular senescence: mitotic clock or culture shock? Cell 2000, 102:407-410
35. Simic-Ogrizovic S, Simic T, Reljic Z, Markovic S, Blagojevic R, Radivojevic D, Lezaic V, Djukanovic L, Mimic-Oka J: Markers of oxidative stress after renal transplantation. Transpl Int 1998, 11 Suppl 1:S125-S129
36. Cristol J P, Vela C, Maggi M F, Descomps B, Mourad G: Oxidative stress and lipid abnormalities in renal transplant recipients with or without chronic rejection. Transplantation 1998, 65:1322-1328
37. Land W, Schneeberger H, Schleibner S, Illner W D, Abendroth D, Rutili G, Arfors K E, Messmer K: The beneficial effect of human recombinant superoxide dismutase on acute and chronic rejection events in recipients of cadaveric renal transplants. Transplantation 1994, 57:211-217
38. Land W: Postischemic reperfusion injury and allograft dysfunction: is allograft rejection the result of a fateful confusion by the immune system of danger and benefit? Transplant Proc 1999, 31:332-336
39. Hancock W W, Buelow R, Sayegh M H, Turka LA: Antibody-induced transplant arteriosclerosis is prevented by graft expression of anti-oxidant and anti-apoptotic genes. Nat Med 1998, 4:1392-1396
   Legend to the Figures:
   FIG. 6:

Tubular atrophy in F344 to LEW renal allografts at day 7, 14, 30, 60 and 100 post Tx according to Banff criteria (0: no tubular atrophy; 1: tubular atrophy in <25% of cortical tubules; 2: tubular atrophy in 26-50% of cortical tubules; 3: tubular atrophy in >51% of cortical tubules). LEW to F344 allografts are shown in day 60 post Tx.

FIG. 7:

Telomere restriction fragment (TRF) length as measured with the TeloREAD assay in normal F344 and LEW kidneys or in F344 and LEW allografts on day 60 (A). Telomere length in F344 to LEW renal allografts in time after transplantation (B). Values are expressed as mean of 3 samples+standard error of the mean (sem).

FIG. 8:

A: Western blot analysis of p21 and actin expression in lysates of normal and F344 to LEW renal allografts (day 7). Samples are representative for 3 samples in each group.

B: Densitometry of p21 Western blots of F344 and LEW renal allografts at various time points after transplantation, corrected for actin content of the samples. Data are expressed as mean+standard error of the mean (sem). (n=3 samples for all groups).

FIG. 9:

A: Immunohistochemical staining of nuclear p16 protein in normal F344 (left) and F344 to LEW allograft at day 60 (middle). No nuclear staining is observed in the absence of the primary antibody (right). (400×)

B: Densitometry of total p16 staining on Western blot of F344 and LEW renal allografts on various time points after transplantation, corrected for actin content of the samples. Data are expressed as mean+standard error of the mean (sem). (n=3 samples for all groups).

FIG. 10:

A: Senescence associated P-galactosidase staining (SA β-gal) (pH 6.0) in frozen sections of normal F344 kidney, F344 to LEW renal allograft at day 60 and a LEW to F344 renal allograft at day 60. Biopsies are representative for 6 biopsies in each group. (×250)

B: Quantification of SA β-gal staining using Zeiss ks400 analysis for normal F344 kidneys, F344 and LEW renal allografts on day 60 post Tx. Data are expressed as mean+standard error of the mean (sem) (n=6 for all groups).

C: Quantification of SA P-gal staining using Zeiss ks400 analysis. F344 to LEW day 7 (n=3), day 14 (n=3), day 30 (n=3), day 60 (n=6) and day 100 (n=3), data expressed as mean+standard error of the mean (sem).

FIG. 11:

A: SA β-gal staining of F344 to LEW allograft (left) and F344 to F344 syngraft (right) on day 60 post Tx. (×250)

B: Telomere restriction fragment (TRF) length of normal F344 and LEW kidneys (n=3) compared with F344 to F344 (day 14 and 60, n=3) and LEW to LEW syngrafts (day 14, n=3), expressed as mean+standard error of the mean (sem).

FIG. 12:

Telomere restriction fragment length of F344 kidneys after 45 minutes of ischemia (n=3) and 45 minutes of ischemia followed by 2 hours of reperfusion (n=3), expressed as mean+standard error of the mean (sem).

FIG. 13:

Schematic representation of events as observed in the F344 to LEW and LEW to F344 renal allografts. Transplantation results in shortening of telomeres in all combinations investigated, this results in activation of p21 and p16. However, only in F344 to LEW renal allografts SA β-gal staining is observed, this suggests that an additional event is required to induce cellular senescence.

1. A method of screening mammalian donor tissues for predisposition to rejection, the method comprising the steps of determining the level of expression of at least one endogenous telomere-binding protein in the donor tissue, and comparing the determined level to a reference level of expression, altered levels of expression in the donor tissue being indicative of a predisposition to rejection.

2. The method of claim 1, wherein the method is carried out on a donor tissue previously removed from a donor body.

3. The method of claim 1, wherein the mammalian tissue is human tissue.

4. The method of claim 1 wherein the tissue is renal tissue.

5. The method of claim 1 wherein the method comprises determining the expression level at least two telomere binding proteins.

6. The method of claim 5 wherein the method comprises determining the expression level of at least three telomere binding proteins.

7. The method of claim 1 wherein the telomere binding protein is selected from the group comprising G22P1, XRCC5, HPOT1, and SIRT2, and their homologues or analogues.

8. The method of claim 1 wherein the telomere binding protein is selected from the group comprising Rif1, Rif2, Rap1, SIRTs 1, 3, 4, 5, 6, 7, 8, ESTL, Est2, TLG1, CDCL3, A26, ATM, HDAC1, hSEP1, hTEP1, HuCds1, MYC, NEK2, p21, PIN2, TNKS, TERC, HTERT, TOP2A, TOP2B, TP53, TRF1, TRF2, and WRN.

9. The method of claim 1 wherein the reference level of expression is determined substantially at the same time as the donor tissue level of expression.

10. The method of claim 1 wherein the reference level of expression is determined from a healthy tissue sample.

11. A kit for screening mammalian donor tissues for predisposition to rejection, the kit comprising reagents for determining the level of expression of at least one endogenous telomere binding protein in the donor tissue.

12. The kit of claim 11, wherein the kit comprises PCR primers for detection of MRNA encoding said telomere binding protein or proteins.

13. The kit of claim 11 wherein said protein or proteins is/are selected from the group comprising G22P1, XRCC5, hPOT1, and SIRT2, or homologues and analogues thereof wherein said kit optionally comprises PCR primers for detection of MRNA encoding said telomere binding protein or proteins.

14. The kit of claim 11 wherein said protein or proteins is/are selected from the group comprising Rif1, Rif2, Rap1, S. IRTSL, 3, 4, 5, 6, 7, 8, Est1, Est2, TLG1, CDCL3, A26, ATM, HDAC1, hSEP1, HTEP1, HuCds1, MYC, NEK2, p21, PIN2, TNKS, TERC, HTERT, TOP2A, TOP2B, TP53, TRF1, TRF2, and WRN wherein said kit optionally comprises PCR primers for detection of MRNA encoding said telomere binding protein or proteins.

15. A method of treatment of a mammalian donor tissue to reduce the risk of rejection, the method comprising the step of treating the tissue with an agent to modulate the activity, half-life or expression level and optionally, the effective functionality of at least one endogenous telomere binding protein.

16. A method of treatment of a mammalian donor tissue to reduce the risk of rejection, the method comprising the step of treating the tissue with an agent to modulate the effective functionality of at least one endogenous telomere binding protein.

17. The method of claim 15, wherein the activity, half-life, expression level, or effective functionality of said at least one protein is enhanced.

18. The method of claim 15, further comprising the step of treating the tissue with an agent to prevent tissue senescence or cell death.

19. The method of claim 18, wherein the agent to prevent tissue senescence comprises a calcineurin inhibitor or an analogue thereof.

20. The method of any of claims 15, wherein said at least one protein is selected from the group comprising G22P1, XRCC5, hPOT1, and SIRT2, and homologues or analogues thereof.

21. The method of claims 15, wherein said at least one protein is selected from the group comprising Rif1, Rif2, Rap1, SIRTSL, 3, 4, 5, 6, 7, 8, Est1, Est2, TLG1, CDCL3, A26, ATM, HDAC1, hSEP1, HTEP1, HuCds1, MYC, NEK2, p21, PIN2, TNKS, TERC, HTERT, TOP2A, TOP2B, TP53, TRF1, TRF2, and WRN.

22. The method of any of claim 15, wherein the treatment of said donor tissue is performed outside the bodies of both the donor and the recipient.

23. (canceled)

24. A non-human mammalian donor tissue in which the expression of at least one endogenous telomere binding protein has been modulated.

25. The tissue of claim 24 wherein the expression of said at least one protein has been enhanced.

26. A method of assessing tissue damage comprising detecting the accumulation of cytological markers of tissue stress.

27. The method of claim 26 wherein the markers are selected from the group comprising senescence associated beta galactosidase (SA beta gal), lipofuscin, advanced glycation end products, and iso-prostanes.

28. The method of claim 2, wherein the mammalian tissue is human tissue.

29. The method of claim 2 wherein the tissue is renal tissue.

30. The method of claim 4 wherein the method comprises determining the expression level at least two telomere binding proteins.

31. The method of claim 4 wherein the telomere binding protein is selected from the group comprising G22P1, XRCC5, HPOT1, and SIRT2, and their homologues or analogues.

32. The method of claim 4 wherein the telomere binding protein is selected from the group comprising Rif1, Rif2, Rap1, SIRTs 1, 3, 4, 5, 6, 7, 8, ESTL, Est2, TLG1, CDCL3, A26, ATM, HDAC1, hSEP1, hTEP1, HuCds1, MYC, NEK2, p21, PIN2, TNKS, TERC, HTERT, TOP2A, TOP2B, TP53, TRF1, TRF2, and WRN.

33. The method of claim 19, wherein said at least one protein is selected from the group comprising G22P1, XRCC5, hPOT1, and SIRT2, and homologues or analogues thereof.

34. The method of claim 19, wherein said at least one protein is selected from the group comprising Rif1, Rif2, Rap1, SIRTSL, 3, 4, 5, 6, 7, 8, Est1, Est2, TLG1, CDCL3, A26, ATM, HDAC1, hSEP1, HTEP1, HuCds1, MYC, NEK2, p21, PIN2, TNKS, TERC, HTERT, TOP2A, TOP2B, TP53, TRF1, TRF2, and WRN.

35. The method of claim 19, wherein the treatment of said donor tissue is performed outside the bodies of both the donor and the recipient.
